(19) United States
(12) Patent Application Publication
(10) Pub. No.: US 2023/0339907 A1
(43) Pub. Date: Oct. 26, 2023

(54) PROCESS FOR THE PREPARATION OF QUATERNIZED PYRIDAZINE DERIVATIVES

(71) Applicant: SYNGENTA CROP PROTECTION AG, Basel (CH)

(72) Inventors: Tomas SMEJKAL, Stein (CH); Raphael DUMEUNIER, Stein (CH); Denis GRIBKOV, Münchwilen (CH)

(73) Assignee: SYNGENTA CROP PROTECTION AG, Basel (CH)

(21) Appl. No.: 18/041,572

(22) PCT Filed: Aug. 13, 2021
(86) PCT No.: PCT/EP2021/072567

§ 371 Date: Feb. 14, 2023

(30) Foreign Application Priority Data
Aug. 14, 2020 (EP) 20191203.7
Jan. 15, 2021 (EP) 21151744.6

(51) Int. Cl. C07C 243/40; C07D 403/04

(52) U.S. Cl. CPC C07D 403/04; C07C 243/40

(57) ABSTRACT

The present invention provides, inter alia, a process for producing a compound of formula (I) wherein the substituents are as defined in claim 1. The present invention further provides intermediate compounds utilised in said process, and methods for producing said intermediate compounds.

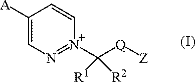

The present invention relates to a novel process for the synthesis of herbicidal pyridazine compounds. Such compounds are known, for example, from WO 2019/034757 and processes for making such compounds or intermediates thereof are also known. Such compounds are typically produced via an alkylation of a pyridazine intermediate.

The alkylation of pyridazine intermediates is known (see for example WO 2019/034757), however, such a process has a number of drawbacks. Firstly, this approach often leads to a non-selective alkylation on either pyridazine nitrogen atom and secondly, an additional complex purification step is required to obtain the desired product. Thus, such an approach is not ideal for large scale production and therefore a new, more efficient synthesis method is desired to avoid the generation of undesirable by-products.

Surprisingly, we have now found that the need for such a non-selective alkylation can be avoided by the use of certain hydrazone intermediates which can be converted to the desired herbicidal pyridazine compounds. Such a process is more convergent and very atom efficient, which may be more cost effective and produce less waste products.

Thus, according to the present invention there is provided a process for the preparation of a compound of formula (I) or an agronomically acceptable salt or zwitterionic species thereof:

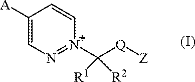

wherein
A is a 6-membered heteroaryl selected from the group consisting of formula A-I to A-VII below

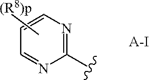

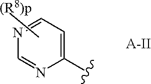

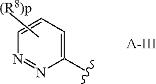

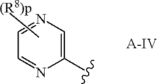

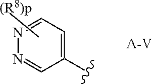

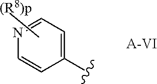

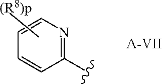

wherein the jagged line defines the point of attachment to the remaining part of a compound of formula (I), p is 0, 1 or 2; and
R¹ is hydrogen or methyl;
R² is hydrogen or methyl;
Q is (CR^{1a}R^{2b})ₘ;
m is 0, 1 or 2;
each R¹^{a} and R^{2b} are independently selected from the group consisting of hydrogen, methyl, —OH and -NH₂;
Z is selected from the group consisting of —CN, —CH₂OR³, —CH(OR⁴)(OR^{4a}), —C(OR⁴)(OR^{4a})(OR^{4b}), —C(O)OR¹⁰, —C(O)NR⁶R⁷ and —S(O)₂OR¹⁰; or
Z is selected from the group consisting of a group of formula Zₐ, Z_{b}, Z_{c}, Z_{d}, Zₑ and Z_{f} below

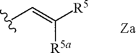

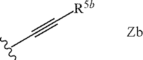

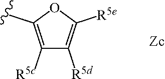

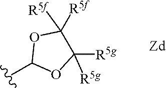

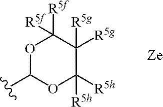

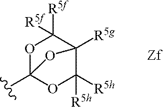

wherein the jagged line defines the point of attachment to the remaining part of a compound of formula (I); and
R³ is hydrogen or —C(O)OR^{10a};
each R⁴, R^{4a} and R^{4b} are independently selected from C₁-C₆alkyl;
each R⁵, R^{5a}, R^{5b}, R^{5c}, R^{5d}, R^{5e}, R^{5f}, R^{5g} and R^{5h} are independently selected from the group consisting of hydrogen and C₁-C₆alkyl;
each R⁶ and R⁷ are independently selected from the group consisting of hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl; and
R^{10a} is selected from the group consisting of hydrogen, C₁-C₆alkyl, phenyl and benzyl;
said process comprising:
reacting a compound of formula (IV);

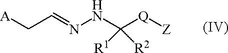

wherein A, Q, Z, R¹ and R² are as defined herein;
with a compound of formula (V) or
a salt or an N-oxide thereof;

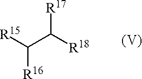

wherein
   each R¹⁵, R¹⁶, R¹⁷ and R¹⁸ are independently selected from the group consisting of halogen, -OR^{15a}, -NR^{16a}R^{17a} and —S(O)₂OR¹⁰; and/or
   R¹⁵ and R¹⁶ together are =O or =NR^{16a} and/or R¹⁷ and R¹⁸ together are =O or =NR^{16a}; or
   R¹⁵ and R¹⁶ together with the carbon atom to which they are attached form a 3- to 6- membered heterocyclyl, which comprises 1 or 2 heteroatoms individually selected from nitrogen and oxygen; or
   R¹⁵ and R¹⁷ together with the carbon atom to which they are attached form a 3- to 6- membered heterocyclyl, which comprises 1 or 2 heteroatoms individually selected from nitrogen and oxygen; and
   each R^{15a} is independently selected from the group consisting of hydrogen and C₁-C₆alkyl;
   each R^{16a} is independently selected from the group consisting of hydrogen and C₁-C₆alkyl;
   each R^{17a} is independently selected from the group consisting of hydrogen and C₁-C₆alkyl;
   to give a compound of formula (I).

According to a second aspect of the invention, there is provided a compound selected from the group consisting of a compound of formula (Ic) and a compound of formula (Id) or an agronomically acceptable salt thereof,

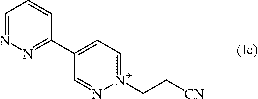 [SMILES: N#CCC[n+]1ccc(-c2cccnn2)cn1]

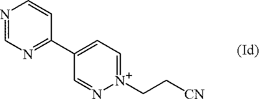 [SMILES: N#CCC[n+]1ccc(-c2ccncn2)cn1]

According to a third aspect of the invention, there is provided an intermediate compound of formula (IV)

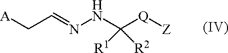

wherein A, Q, Z, R¹ and R² are as defined herein.

According to a fourth aspect of the invention, there is provided the use of a compound of formula (II) for preparing a compound of formula (I)

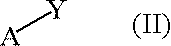

wherein A and Y are as defined herein.

According to a fifth aspect of the invention, there is further provided an intermediate compound of formula (II-a)

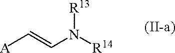

wherein A is a 6-membered heteroaryl selected from the group consisting of formula A-I, A-II, A-III, A-IV, A-V and A-VII below

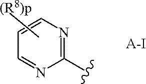

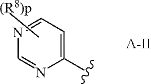

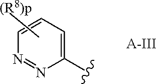

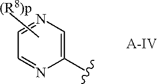

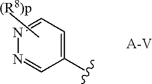

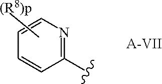

wherein the jagged line defines the point of attachment to the remaining part of a compound of formula (I), p and R⁸ are as defined herein;
R¹³ and R¹⁴ are independently selected from the group consisting of C₂-C₆alkyl, C₁-C₆haloalkyl and phenyl; or
R¹³ and R¹⁴ together with the nitrogen atom to which they are attached form a 4- to 6-membered heterocyclyl ring which optionally comprises one additional heteroatom individually selected from nitrogen, oxygen and sulfur.

According to a sixth aspect of the invention, there is provided the use of a compound of formula (VI) for preparing a compound of formula (I)

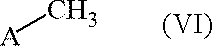

wherein A is as defined herein.

According to a seventh aspect of the invention, there is provided the use of a compound of formula (III) for preparing a compound of formula (I)

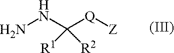

wherein R¹, R², Q and Z are as defined herein.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl, ethyl, n-propyl, 1-methylethyl (iso-propyl), n-butyl, and 1-dimethylethyl (t-butyl).

As used herein, the term “C₁-C₆alkoxy” refers to a radical of the formula —ORₐ where Rₐ is a C₁-C₆alkyl radical as generally defined above. Examples of C₁-C₆alkoxy include, but are not limited to, methoxy, ethoxy, propoxy, iso-propoxy and t-butoxy.

The process of the present invention can be carried out in separate process steps, wherein the intermediate compounds can be isolated at each stage. Alternatively, the process can be carried out in a one-step procedure wherein the intermediate compounds produced are not isolated. Thus, it is possible for the process of the present invention to be conducted in a batch wise or continuous fashion.

The compounds of formula (I) will typically be provided in the form of an agronomically acceptable salt, a zwitterion or an agronomically acceptable salt of a zwitterion. This invention covers processes to make all such agronomically acceptable salts, zwitterions and mixtures thereof in all proportions.

For example a compound of formula (I) wherein Z comprises an acidic proton, may exist as a zwitterion, a compound of formula (I-I), or as an agronomically acceptable salt, a compound of formula (I-II) as shown below:

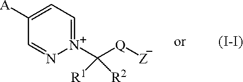

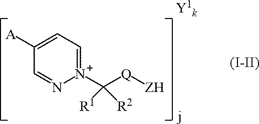

wherein, Y¹ represents an agronomically acceptable anion and j and k represent integers that may be selected from 1, 2 or 3, dependent upon the charge of the respective anion Y¹.

A compound of formula (I) may also exist as an agronomically acceptable salt of a zwitterion, a compound of formula (I-III) as shown below:

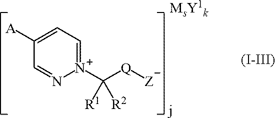

wherein, Y¹ represents an agronomically acceptable anion, M represents an agronomically acceptable cation (in addition to the pyridazinium cation) and the integers j, k and s may be selected from 1, 2 or 3, dependent upon the charge of the respective anion Y¹ and respective cation M.

Suitable agronomically acceptable salts of the present invention, represented by an anion Y¹, include but are not limited chloride, bromide, iodide, fluoride, 2-naphthalenesulfonate, acetate, adipate, methoxide, ethoxide, propoxide, butoxide, aspartate, benzenesulfonate, benzoate, bicarbonate, bisulfate, bitartrate, butylsulfate, butylsulfonate, butyrate, camphorate, camsylate, caprate, caproate, caprylate, carbonate, citrate, diphosphate, edetate, edisylate, enanthate, ethanedisulfonate, ethanesulfonate, ethylsulfate, formate, fumarate, gluceptate, gluconate, glucoronate, glutamate, glycerophosphate, heptadecanoate, hexadecanoate, hydrogen sulfate, hydroxide, hydroxynaphthoate, isethionate, lactate, lactobionate, laurate, malate, maleate, mandelate, mesylate, methanedisulfonate, methylsulfate, mucate, myristate, napsylate, nitrate, nonadecanoate, octadecanoate, oxalate, pelargonate, pentadecanoate, pentafluoropropionate, perchlorate, phosphate, propionate, propylsulfate, propylsulfonate, succinate, sulfate, tartrate, tosylate, tridecylate, triflate, trifluoroacetate, undecylinate and valerate.

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium.

Preferred compounds of formula (I), wherein Z comprises an acidic proton, can be represented as either (I-I) or (I-II). For compounds of formula (I-II) emphasis is given to salts when Y¹ is chloride, bromide, iodide, hydroxide, bicarbonate, acetate, pentafluoropropionate, triflate, trifluoroacetate, methylsulfate, tosylate, benzoate and nitrate, wherein j and k are 1. Preferably, Y¹ is chloride, bromide, iodide, hydroxide, bicarbonate, acetate, trifluoroacetate, methylsulfate, tosylate and nitrate, wherein j and k are 1. More preferably, Y¹ is chloride or bromide, wherein j and k are 1. Most preferably, Y¹ is chloride, wherein j and k are 1.

Thus where a compound of formula (I) is drawn in protonated form herein, the skilled person would appreciate that it could equally be represented in unprotonated or salt form with one or more relevant counter ions.

Compounds of formula (I) wherein m is 0 may be represented by a compound of formula (I-Ia) as shown below:

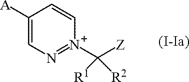

wherein R¹, R², A and Z are as defined for compounds of formula (I).

Compounds of formula (I) wherein m is 1 may be represented by a compound of formula (I-Ib) as shown below:

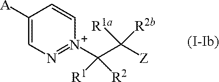

wherein R¹, R², R^{1a}, R^{2b}, A and Z are as defined for compounds of formula (I).

Compounds of formula (I) wherein m is 2 may be represented by a compound of formula (I-Ic) as shown below:

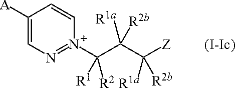

wherein R¹, R², R^{1a}, R^{2b}, A and Z are as defined for compounds of formula (I).

Compounds of formula (II) wherein Y is Y-I may be represented by a compound of formula (II-a) as shown below:

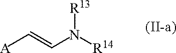

wherein A, R¹³ and R¹⁴ are as defined herein.

Compounds of formula (II) wherein Y is Y-II may be represented by a compound of formula (II-b) as shown below:

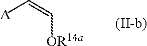

wherein A and R^{14a} are as defined herein.

Compounds of formula (II) wherein Y is Y-III may be represented by a compound of formula (II-c) as shown below:

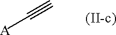

wherein A is as defined herein.

The skilled person would appreciate that where in a compound of formula (II-b) R^{14a} is hydrogen, it could equally be represented in unprotonated or salt form with one or more relevant counter ions. For a compound of formula (II-Ib), (II-IIb) or (II-VIIb) wherein R^{14a} is hydrogen emphasis is given to calcium, cesium, lithium, magnesium, potassium, sodium and zinc salts.

The skilled person would appreciate that the compound of formula (IV) may exist as E and/or Z isomers. This invention covers all such isomers and mixtures thereof in all proportions.

For example, a compound of formula (IV) can be drawn in at least 2 different isomeric forms (a compound of formula (IV) or (IVa)) as shown below. Moreover, the individual isomers, or intermediates depicted below may interconvert in solid state, in solution, or under exposure to light.

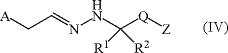

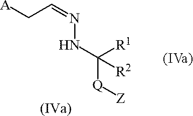

The following list provides definitions, including preferred definitions, for substituents m, p, A, Q, Y, Z, Z², R¹, R², R^{1a}, R^{2b}, R³, R⁴, R^{4a}, R^{4b}, R⁵, R^{5a}, R^{5b}, R^{5c}, R^{5d}, R^{5e}, R^{5f}, R^{5g}, R^{5h}, R⁶, R⁷, R⁸, R¹⁰, R^{10a}, R¹³, R¹⁴, R^{14a}, R^{14b}, R¹⁵, R^{15a}, R¹⁶, R^{16a}, R¹⁷, R^{17a}, R¹⁸, R²², R²³, R²⁴, R²⁵, R²⁶ with reference to the process according to the invention. For any one of these substituents, any of the definitions given below may be combined with any definition of any other substituent given below or elsewhere in this document.

A is a 6-membered heteroaryl selected from the group consisting of formula A-I to A-VII below

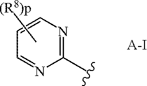

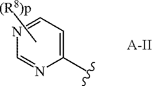

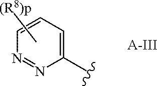

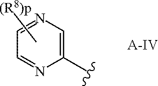

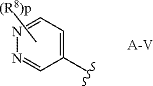

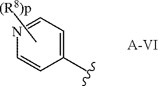

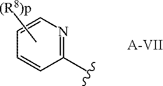

wherein the jagged line defines the point of attachment to the remaining part of a compound of formula (I), p is 0, 1 or 2 (preferably, p is 0 or 1, more preferably, p is 0).

Preferably, A is a 6-membered heteroaryl selected from the group consisting of formula A-I, A-II, A-III, A-IV, A-V and A-VII below

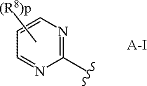

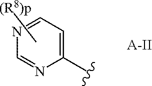

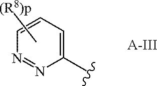

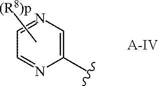

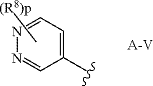

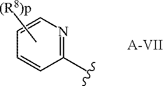

wherein the jagged line defines the point of attachment to the remaining part of a compound of formula (I), p is 0, 1 or 2 (preferably, p is 0 or 1, more preferably, p is 0).

More preferably, A is a 6-membered heteroaryl selected from the group consisting of formula A-la, A-IIa, A-IIIa, A-IVa, A-Va and A-VIIa below

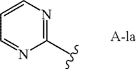

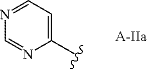

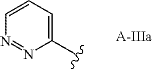

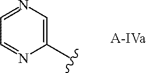

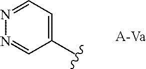

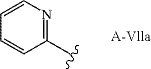

wherein the jagged line defines the point of attachment to the remaining part of a compound of formula (I).

Even more preferably, A is selected from the group consisting of formula A-la to A-IIIa below,

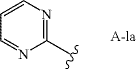

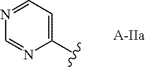

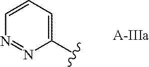

wherein the jagged line defines the point of attachment to the remaining part of a compound of formula (I).

Most preferably, A is the group A-Ia or A-IIIa.

R¹ is hydrogen or methyl, preferably R¹ is hydrogen.

R² is hydrogen or methyl, preferably R² is hydrogen.

In a preferred embodiment R¹ and R² are hydrogen.

Q is (CR^{1a}R^{2b})ₘ. Preferably, Q is CH₂.

m is 0, 1 or 2, preferably m is 1 or 2. Most preferably, m is 1.

each R¹^{a} and R^{2b} are independently selected from the group consisting of hydrogen, methyl, —OH and —NH₂. More preferably, each R¹^{a} and R^{2b} are independently selected from the group consisting of hydrogen and methyl. Most preferably R¹^{a} and R^{2b} are hydrogen.

Z is selected from the group consisting of —CN, —CH₂OR³, —CH(OR⁴)(OR^{4a}), —C(OR⁴)(OR^{4a})(OR^{4b}), —C(O)OR¹⁰, —C(O)NR⁶R⁷ and —S(O)₂OR¹⁰. Preferably, Z is selected from the group consisting of —CN, —CH₂OR³, —C(O)OR¹⁰, —C(O)NR⁶R⁷ and —S(O)₂OR¹⁰. More preferably, Z is selected from the group consisting of —CN, —CH₂OH, —C(O)OR¹⁰, —C(O)NH₂ and —S(O)₂OR¹⁰. Even more preferably, Z is selected from the group consisting of —CN, —CH₂OH, —C(O)OR¹⁰ and —S(O)₂OR¹⁰. Yet even more preferably still, Z is selected from the group consisting of —CN, —C(O)OR¹⁰ and —S(O)₂OR¹⁰. Yet even more preferably still, Z is selected from the group consisting of —CN, —C(O)OCH₂CH₃, —C(O)OC(CH₃)₃, —C(O)OH, —S(O)₂OCH₂C(CH₃)₃ and —S(O)₂OH. Yet further more preferably still, Z is selected from the group consisting of —CN, —C(O)OCH₂CH₃, —C(O)OC(CH₃)₃ and —C(O)OH. Most preferably, Z is —CN or —C(O)OC(CH₃)₃.

In an alternative embodiment Z is selected from the group consisting of a group of formula Zₐ, Z_{b}, Z_{c}, Z_{d}, Zₑ and Z_{f} below

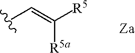

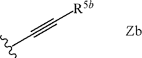

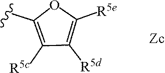

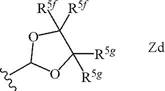

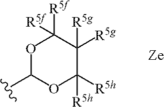

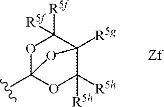

wherein the jagged line defines the point of attachment to the remaining part of a compound of formula (I). Preferably, Z is selected from the group consisting of a group of formula Zₐ, Z_{b}, Z_{d}, Zₑ and Z_{f}. More preferably, Z is selected from the group consisting of a group of formula Zₐ, Z_{d} and Zₑ.

In another embodiment of the invention Z is —C(O)OR¹⁰ and R¹⁰ is hydrogen or C₁-C₆alkyl. Preferably Z is —C(O)OC(CH₃)₃.

In another embodiment of the invention Z is selected from the group consisting of —CN, —CH₂OH, —C(O)OR¹⁰ and —S(O)₂OR¹⁰, or Z is selected from the group consisting of a group of formula Zₐ, Z_{d} and Zₑ. Preferably, Z is selected from the group consisting of —CN, —CH₂OH, —C(O)OR¹⁰, —S(O)₂OR¹⁰ and —CH═CH₂. More preferably, Z is —CN or —C(O)OR¹⁰.

The skilled person would appreciate that Z² below is a subset of Z for specific embodiments of the invention.

Z² is —C(O)OH or —S(O)₂OH. Preferably, Z² is —C(O)OH.

R³ is hydrogen or —C(O)OR^{10a}. Preferably, R³ is hydrogen.

Each R⁴, R^{4a} and R^{4b} are independently selected from C₁-C₆alkyl. Preferably, each R⁴, R^{4a} and R^{4b} are methyl.

Each R⁵, R^{5a}, R^{5b}, R^{5c}, R^{5d}, R^{5e}, R^{5f}, R^{5g} and R^{5h} are independently selected from the group consisting of hydrogen and C₁-C₆alkyl. More preferably, each R⁵, R^{5a}, R^{5b}, R^{5c}, R^{5d}, R^{5e}, R^{5f}, R^{5g} and R^{5h} are independently hydrogen or methyl. Most preferably, each R⁵, R^{5a}, R^{5b}, R^{5c}, R^{5d}, R^{5e}, R^{5f}, R^{5g} and R^{5h} are hydrogen.

Each R⁶ and R⁷ are independently selected from the group consisting of hydrogen and C₁-C₆alkyl. Preferably, each R⁶ and R⁷ are independently hydrogen or methyl. Most preferably, each R⁶ and R⁷ are hydrogen.

Each R⁸ is independently selected from the group consisting of halo, —NH₂, methyl and methoxy. Preferably, R⁸ is halo (preferably, chloro or bromo) or methyl. More preferably, R⁸ is halo (preferably, chloro or bromo).

R¹⁰ is selected from the group consisting of hydrogen, C₁-C₆alkyl, phenyl and benzyl. Preferably, R¹⁰ is selected from the group consisting of hydrogen and C₁-C₆alkyl. More preferably, R¹⁰ is selected from the group consisting of hydrogen, methyl, ethyl, iso-propyl, 2,2-dimethylpropyl and tert-butyl.

R^{10a} is selected from the group consisting of hydrogen, C₁-C₆alkyl, phenyl and benzyl. Preferably, R^{10a} is selected from the group consisting of hydrogen, C₁-C₆alkyl and phenyl. More preferably, R^{10a} is selected from the group consisting of hydrogen and C₁-C₆alkyl.

In one embodiment of the invention, R¹⁰ is ethyl or tert-butyl. Preferably, R¹⁰ is tert-butyl.

Y is selected from the group consisting of a group of formula Y-I, Y-II and Y-III below

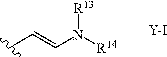

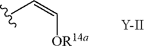

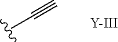

wherein the jagged line defines the point of attachment to the remaining part of a compound of formula (II).

Preferably, Y is the group Y-I below

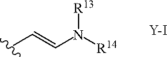

wherein the jagged line defines the point of attachment to the remaining part of a compound of formula (II).

R¹³ and R¹⁴ are independently selected from the group consisting of hydrogen, C₁-C₆alkyl, C₁-C₆haloalkyl and phenyl. Preferably, R¹³ and R¹⁴ are independently selected from the group consisting of hydrogen and C₁-C₆alkyl. More preferably, R¹³ and R¹⁴ are independently selected from the group consisting of hydrogen, methyl and ethyl. Even more preferably, R¹³ and R¹⁴ are independently hydrogen or methyl. Most preferably, R¹³ and R¹⁴ are methyl.

Alternatively, R¹³ and R¹⁴ together with the nitrogen atom to which they are attached form a 4- to 6-membered heterocyclyl ring which optionally comprises one additional heteroatom individually selected from nitrogen, oxygen and sulfur. Preferably, R¹³ and R¹⁴ together with the nitrogen atom to which they are attached form a 4- to 6-membered heterocyclyl ring which optionally comprises one additional heteroatom individually selected from nitrogen and oxygen. More preferably, R¹³ and R¹⁴ together with the nitrogen atom to which they are attached form a 5- to 6-membered heterocyclyl ring which optionally comprises one additional heteroatom individually selected from nitrogen and oxygen. Even more preferably, R¹³ and R¹⁴ together with the nitrogen atom to which they are attached form a 5- to 6-membered heterocyclyl ring which optionally comprises one additional oxygen atom. Most preferably, R¹³ and R¹⁴ together with the nitrogen atom to which they are attached form a morpholinyl, piperidinyl or pyrrolidinyl group.

R^{14a} is selected from the group consisting of hydrogen (or salt thereof), C₁-C₆alkyl and —C(O)R^{14b}. Preferably, R^{14a} is selected from the group consisting of hydrogen (or salt thereof) and C₁-C₆alkyl. More preferably, R^{14a} is selected from the group consisting of hydrogen (or salt thereof), methyl and ethyl. Most preferably, R^{14a} is hydrogen (or salt thereof).

R^{14b} is selected from the group consisting of hydrogen, C₁-C₆alkyl and C₁-C₆haloalkyl. Preferably R^{14b} is C₁-C₆alkyl.

Each R¹⁵, R¹⁶, R¹⁷ and R¹⁸ are independently selected from the group consisting of halogen, -OR^{15a}, -NR^{16a}R^{17a} and —S(O)₂OR¹⁰. Preferably, each R¹⁵, R¹⁶, R¹⁷ and R¹⁸ are independently selected from the group consisting of halogen, -OR^{15a} and -NR^{16a}R^{17a}. More preferably, each R¹⁵, R¹⁶, R¹⁷ and R¹⁸ are independently selected from the group consisting of -OR^{15a} and -NR^{16a}R^{17a}. Even more preferably, each R¹⁵, R¹⁶, R¹⁷ and R¹⁸ are independently selected from -OR^{15a}.

Alternatively, R¹⁵ and R¹⁶ together are =O or =NR^{16a} and/or R¹⁷ and R¹⁸ together are =O or =NR^{16a.} Preferably, R¹⁵ and R¹⁶ together are =O and/or R¹⁷ and R¹⁸ together are =O. Most preferably, R¹⁵ and R¹⁶ together are =O and R¹⁷ and R¹⁸ together are =O.

Alternatively, R¹⁵ and R¹⁶ together with the carbon atom to which they are attached form a 3- to 6-membered heterocyclyl, which comprises 1 or 2 heteroatoms individually selected from nitrogen and oxygen. Preferably, R¹⁵ and R¹⁶ together with the carbon atom to which they are attached form a 5- to 6- membered heterocyclyl, which comprises 1 or 2 heteroatoms individually selected from nitrogen and oxygen. More preferably, R¹⁵ and R¹⁶ together with the carbon atom to which they are attached form a 6- membered heterocyclyl, which comprises 2 oxygen heteroatoms.

Alternatively, R¹⁵ and R¹⁷ together with the carbon atom to which they are attached form a 3- to 6-membered heterocyclyl, which comprises 1 or 2 heteroatoms individually selected from nitrogen and oxygen. Preferably, R¹⁵ and R¹⁷ together with the carbon atom to which they are attached form a 5- to 6- membered heterocyclyl, which comprises 1 or 2 heteroatoms individually selected from nitrogen and oxygen. More preferably, R¹⁵ and R¹⁷ together with the carbon atom to which they are attached form a 6- membered heterocyclyl, which comprises 2 oxygen heteroatoms.

Each R^{15a} is independently selected from the group consisting of hydrogen and C₁-C₆alkyl. Preferably, each R^{15a} is independently hydrogen or methyl.

Each R^{16a} is independently selected from the group consisting of hydrogen and C₁-C₆alkyl. Preferably, each R^{16a} is independently hydrogen or methyl.

Each R^{17a} is independently selected from the group consisting of hydrogen and C₁-C₆alkyl. Preferably, each R^{17a} is independently hydrogen or methyl.

In one embodiment of the invention the compound of formula (V) is a compound selected from the group consisting of a compound of formula (Va), (Vb), (Vc), (Vd), (Ve), (Vf), (Vg), (Vh), (Vj), (Vk) and (Vm),

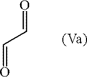

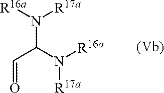

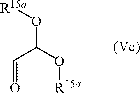

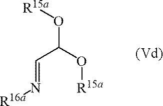

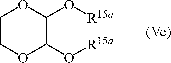

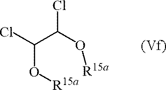

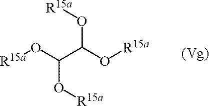

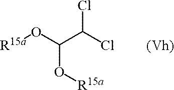

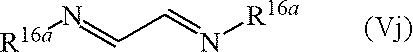

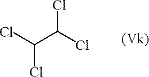

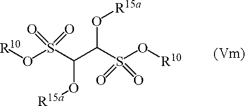

wherein each R¹⁰, R^{15a}, R^{16a} and R^{17a} are as defined herein.

Preferably, the compound of formula (V) is a compound selected from the group consisting of a compound of formula (Va), (Vb), (Vc), (Vd), (Ve), (Vf), (Vg) and (Vh),

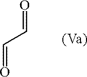

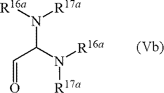

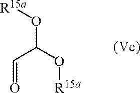

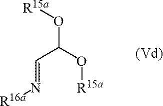

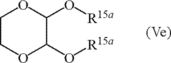

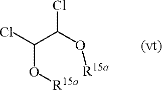

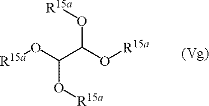

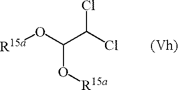

wherein each R^{15a,} R^{16a} and R^{17a} are as defined herein.

More preferably, the compound of formula (V) is a compound selected from the group consisting of a compound of formula (Va), (Vc), (Ve), (Vf) and (Vg),

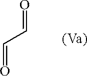

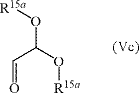

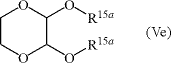

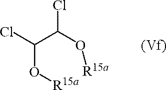

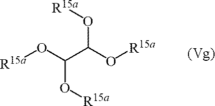

wherein each R^{15a} are as defined herein.

Even more preferably, the compound of formula (V) is a compound selected from the group consisting of a compound of formula (Va), (Vc-I), (Vc-II), (Ve-I), (Ve-II), (Vf-I) and (Vg-I),

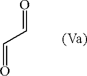

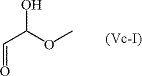

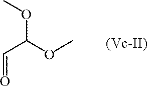

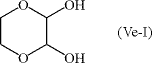

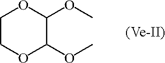

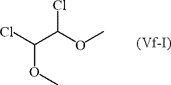

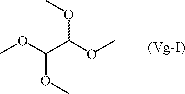

Even more preferably still, the compound of formula (V) is a compound selected from the group consisting of a compound of formula (Va), (Vc-II), (Ve-I), (Vf-I) and (Vg-I),

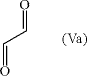

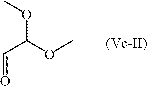

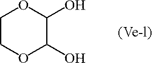

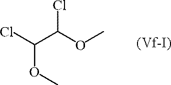

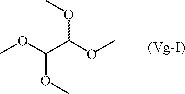

Most preferably, the compound of formula (V) is a compound of formula (Va)

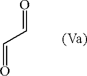

Preferably, the compound of formula (I) is further subjected to a hydrolysis, oxidation and/or a salt exchange (i.e converted) to give an agronomically acceptable salt of formula (la) or a zwitterion of formula (Ib),

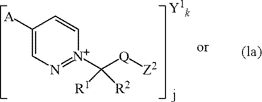

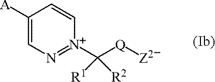

wherein Y¹ represents an agronomically acceptable anion and j and k represent integers that may be selected from 1, 2 or 3 (preferably, Y¹ is chloride or bromide and j and k are 1, more preferably, Y¹ is chloride and j and k are 1), and A, R¹, R² and Q are as defined herein and Z² is —C(O)OH or —S(O)₂OH (the skilled person would appreciate that Z² represents —C(O)O⁻ or —S(O)₂O).

More preferably, the the compound of formula (I) is further subjected to a hydrolysis, oxidation and/or a salt exchange (i.e converted) to give a compound of formula (la),

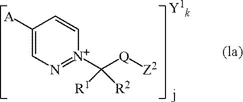

wherein Y¹ represents an agronomically acceptable anion and j and k represent integers that may be selected from 1, 2 or 3 (preferably, Y¹ is chloride or bromide and j and k are 1, more preferably, Y¹ is chloride (Cl⁻) and j and k are 1), and A, R¹, R² and Q are as defined herein and Z² is —C(O)OH.

Where a compound of formula (I) is drawn in protonated form herein (R¹⁰ is hydrogen), the skilled person would appreciate that it could equally be represented in unprotonated or salt form with one or more relevant counter ions.

Preferably, in a compound of formula (la) Y¹ is chloride, bromide, iodide, hydroxide, bicarbonate, acetate, trifluoroacetate, methylsulfate, tosylate, benzoate and nitrate, wherein j and k are 1. More preferably, in a compound of formula (la) Y¹ is chloride (Cl⁻) or bromide (Br) and j and k are 1. Most preferably, in a compound of formula (la) Y¹ is chloride (Cl⁻) and j and k are 1.

The present invention further provides an intermediate compound of formula (IV)

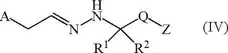

wherein A, Q, Z, R¹ and R² are as defined herein.

Preferably, in an intermediate compound of formula (IV), A is a 6-membered heteroaryl selected from the group consisting of formula A-la, A-IIa, and A-IIIa below

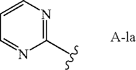

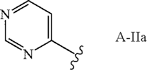

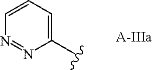

wherein the jagged line defines the point of attachment to the remaining part of a compound of formula (V) (preferably, A is the group A-la or A-IIIa);
R¹ and R² are hydrogen;
Q is (CR^{1a}R^{2b})_{m;}
m is 1;
R^{1a} and R^{2b} are hydrogen;
Z is —CN, —CHzOH, —C(O)OR¹⁰, —S(O)₂OR¹⁰ or —CH═CH₂ (preferably, Z is —CN or —C(O)OR¹⁰); and R¹⁰ is selected from the group consisting of hydrogen, C₁-C₆alkyl, phenyl and benzyl (preferably, R¹⁰ is hydrogen or C₁-C₆alkyl).

More preferably, the intermediate compound of formula (IV) is selected from the group consisting of a compound of formula (IV-I), (IV-II), (IV-III), (IV-IV), (IV-V), (IV-VI), (IV-VII), (IV-VIII), (IV-IX), (IV-X) (IV-XI), (IV-XII), (IV-XIII), (IV-XIV) and (IV-XV) below,

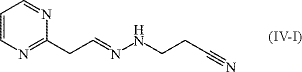 [SMILES: N#CCCN/N=C/Cc1ncccn1]

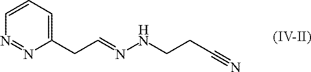 [SMILES: N#CCCN/N=C/Cc1cccnn1]

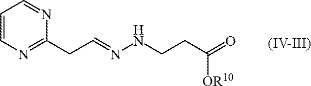

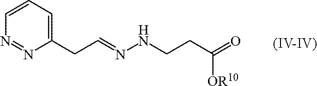

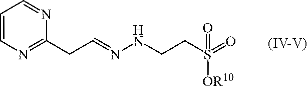

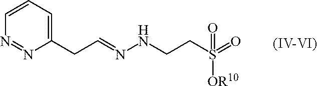

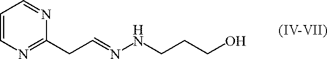 [SMILES: OCCCN/N=C/Cc1ncccn1]

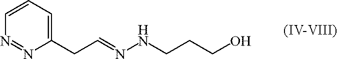 [SMILES: OCCCN/N=C/Cc1cccnn1]

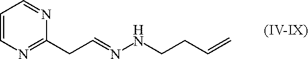

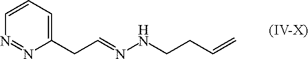

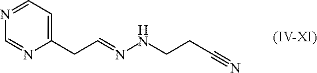 [SMILES: N#CCCN/N=C/Cc1ccncn1]

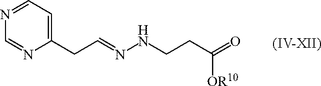

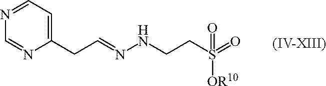

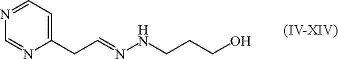 [SMILES: OCCCN/N=C/Cc1ccncn1]

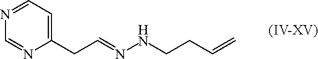

Even more preferably, the intermediate compound of formula (IV) is selected from the group consisting of a compound of formula (IV-I), (IV-II), (IV-III), (IV-IV), (IV-V), (IV-VI), (IV-VII), (IV-VIII), (IV-IX) and (IV-X) below,

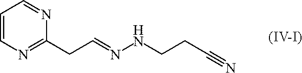 [SMILES: N#CCCN/N=C/Cc1ncccn1]

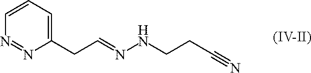 [SMILES: N#CCCN/N=C/Cc1cccnn1]

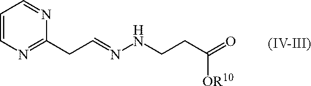

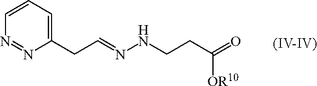

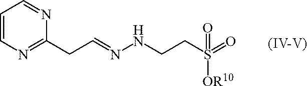

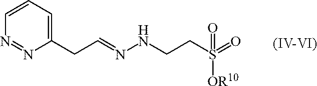

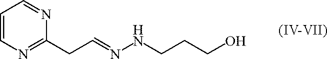 [SMILES: OCCCN/N=C/Cc1ncccn1]

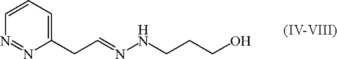 [SMILES: OCCCN/N=C/Cc1cccnn1]

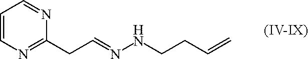

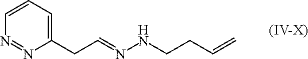

Even more preferably still, the intermediate compound of formula (IV) is selected from the group consisting of a compound of formula (IV-I), (IV-II), (IV-a), (IV-b), (IV-c) and (IV-d) below,

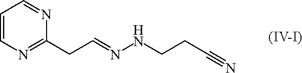 [SMILES: N#CCCN/N=C/Cc1ncccn1]

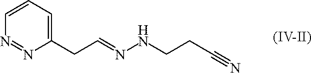 [SMILES: N#CCCN/N=C/Cc1cccnn1]

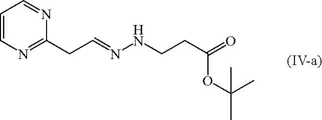 [SMILES: CC(C)(C)OC(=O)CCN/N=C/Cc1ncccn1]

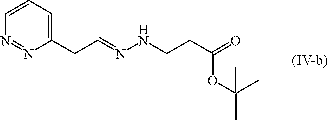 [SMILES: CC(C)(C)OC(=O)CCN/N=C/Cc1cccnn1]

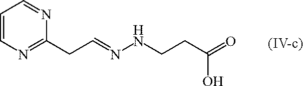 [SMILES: O=C(O)CCN/N=C/Cc1ncccn1]

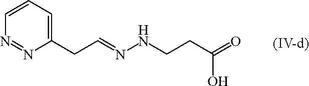 [SMILES: O=C(O)CCN/N=C/Cc1cccnn1]

The present invention further provides an intermediate compound of formula (ll-a)

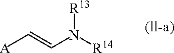

wherein A is a 6-membered heteroaryl selected from the group consisting of formula A-I, A-II, A-III, A-IV, A-V and A-VII below

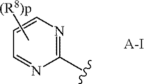

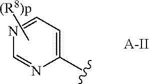

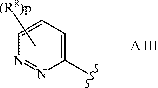

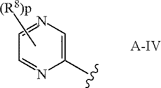

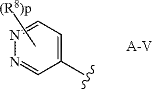

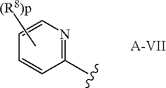

wherein the jagged line defines the point of attachment to the remaining part of a compound of formula (I), p and R⁸ are as defined herein; and
R¹³ and R¹⁴ are independently selected from the group consisting of C₂-C₆alkyl, C₁-C₆haloalkyl and phenyl; or
R¹³ and R¹⁴ together with the nitrogen atom to which they are attached form a 4- to 6-membered heterocyclyl ring which optionally comprises one additional heteroatom individually selected from nitrogen, oxygen and sulfur.

Preferably, in an intermediate compound of formula (ll-a),
A is a 6-membered heteroaryl selected from the group consisting of formula A-la, A-Ila, and A-IIIa below

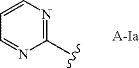

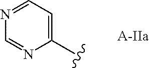

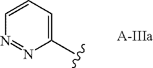

wherein the jagged line defines the point of attachment to the remaining part of a compound of formula (ll-a) (preferably, A is the group A-la or A-IIIa); and
R¹³ and R¹⁴ are independently selected from C₂-C₆alkyl; or
R¹³ and R¹⁴ together with the nitrogen atom to which they are attached form a 4- to 6-membered heterocyclyl ring which optionally comprises one additional oxygen heteroatom (preferably, R¹³ and R¹⁴ together with the nitrogen atom to which they are attached form a morpholinyl, piperidinyl or pyrrolidinyl group).

More preferably, the compound of formula (II-a) is selected from the group consisting of a compound of formula (ll-la), (II-Ila), (II-IIIa), (II-IVa), (II-Va), (II-VIa), (II-VIIa), (II-VIIIa) and (II-IXa) below,

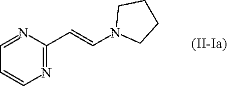 [SMILES: C(=C\N1CCCC1)/c1ncccn1]

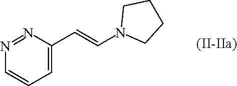 [SMILES: C(=C\N1CCCC1)/c1cccnn1]

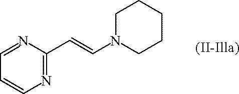 [SMILES: C(=C\N1CCCCC1)/c1ncccn1]

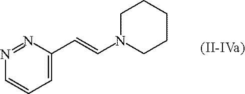 [SMILES: C(=C\N1CCCCC1)/c1cccnn1]

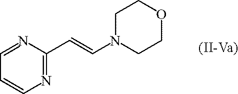 [SMILES: C(=C\N1CCOCC1)/c1ncccn1]

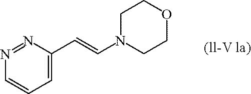 [SMILES: C(=C\N1CCOCC1)/c1cccnn1]

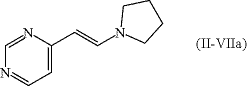 [SMILES: C(=C\N1CCCC1)/c1ccncn1]

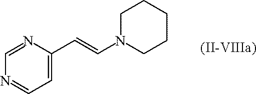 [SMILES: C(=C\N1CCCCC1)/c1ccncn1]

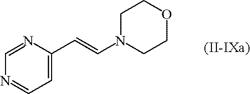 [SMILES: C(=C\N1CCOCC1)/c1ccncn1]

Even more preferably, the compound of formula (ll-a) is selected from the group consisting of a compound of formula (II-Ia), (II-IIa), (ll-llla), (II-IVa), (II-Va) and (II-Vla) below,

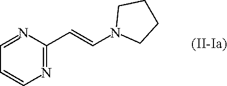 [SMILES: C(=C\N1CCCC1)/c1ncccn1]

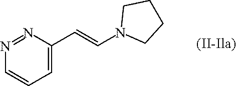 [SMILES: C(=C\N1CCCC1)/c1cccnn1]

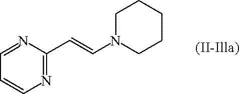 [SMILES: C(=C\N1CCCCC1)/c1ncccn1]

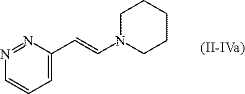 [SMILES: C(=C\N1CCCCC1)/c1cccnn1]

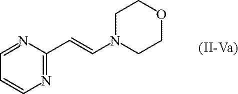 [SMILES: C(=C\N1CCOCC1)/c1ncccn1]

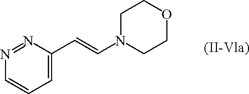 [SMILES: C(=C\N1CCOCC1)/c1cccnn1]

In an alternative embodiment of the invention the compound of formula (II-a) is a compound selected from the group consisting of a compound of formula (II-laa), (II-Ilaa) and (II-IIIaa) below

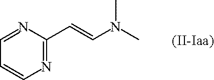

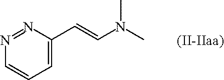

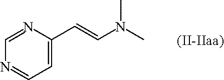

In one embodiment of the invention there is provided the use of a compound of formula (II-b) (or a salt thereof) for preparing a compound of formula (I)

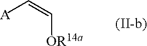

wherein A and R^{14a} are as defined herein.

Preferably, there is provided the use of a compound of formula (II-b) (or a salt thereof) for preparing a compound of formula (I) wherein
A is selected from the group consisting of formula A-la to A-IIIa (preferably, A-la or A-IIIa) below,

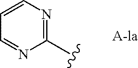

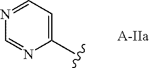

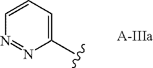

wherein the jagged line defines the point of attachment to the remaining part of a compound of formula (II-b); and
R^{14a} is hydrogen.

More preferably, there is provided the use of a compound of formula (II-Ib), (ll-llb), (II-IIIb), (II-IVb), (II-Vb), (II-Vlb), (II-Vllb), (II-VIlIb) or (II-IXb) below

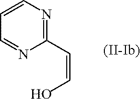

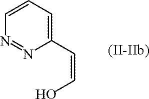

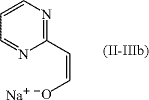

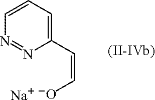

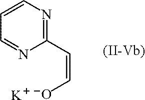

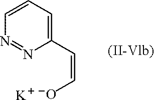

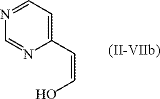

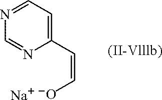

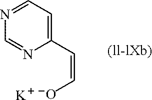

for preparing a compound of formula (I).

Even more preferably, there is provided the use of a compound of formula (II-Ib), (II-IIb), (II-IIIb), (II-IVb), (II-Vb) or (II-VIb) below

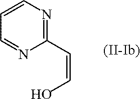

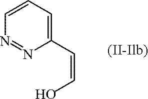

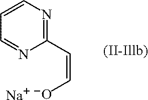

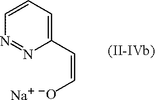

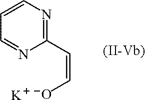

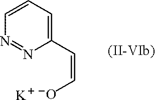

for preparing a compound of formula (I).

In another embodiment of the invention there is provided the the use of a compound of formula (II-c) for preparing a compound of formula (I)

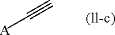

wherein A is as defined herein.

Preferably, there is provided the use of a compound selected from the group consisting of a compound of formula (II-Ic), (ll-llc) and (II-Illc) below

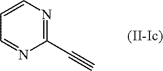

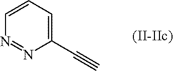

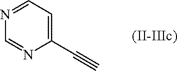

for preparing a compound of formula (I).

More preferably, there is provided the use of a compound of formula (II-Ic) or (ll-llc) below

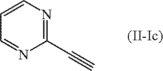

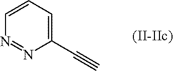

for preparing a compound of formula (I).

In one embodiment of the invention there is provided the use of a compound of formula (VI) for preparing a compound of formula (I)

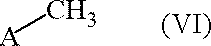

wherein A is as defined herein.

Preferably, there is provided the use of a compound of formula (VI-I), (VI-II) or (VI-III) below

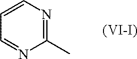

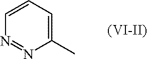

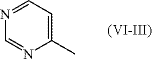

for preparing a compound of formula (I).

More preferably, there is provided the use of a compound of formula (VI-I) or a compound of formula (VI-II) below

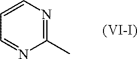

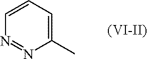

for preparing a compound of formula (I).

Compounds of formula (VI) are are either known in the literature or may be prepared by known literature methods.

The present invention further provides a process as referred to above, wherein the compound of formula (IV) is produced by reacting a compound of formula (II):

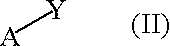

wherein A is as defined herein;

Y is selected from the group consisting of a group of formula Y-I, Y-II and Y-III below

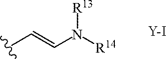

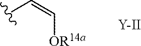

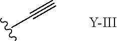

R¹³ and R¹⁴ are independently selected from the group consisting of hydrogen, C₁-C₆alkyl, C₁-C₆haloalkyl and phenyl; or
R¹³ and R¹⁴ together with the nitrogen atom to which they are attached form a 4- to 6-membered heterocyclyl ring which optionally comprises one additional heteroatom individually selected from nitrogen, oxygen and sulfur; and
R^{14a} is selected from the group consisting of hydrogen, C₁-C₆alkyl and -C(O)R^{14b};
R^{14b} is selected from the group consisting of hydrogen, C₁-C₆alkyl and C₁-C₆haloalkyl;
with a compound of formula (III):

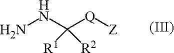

wherein R¹, R², Q and Z are as defined herein, to give a compound of formula (IV);

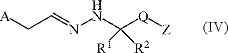

wherein A, Q, Z, R¹ and R² are as defined herein.

The present invention further provides a process as referred to above, wherein the compound of formula (II-a), is produced by: reacting a compound of formula (VI)

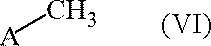

wherein A is as defined herein, with a compound of formula (VII)

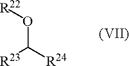

wherein R²² is C₁-C₆alkyl (preferably, methyl);
R²³ and R²⁴ are independently selected from the group consisting of C₁-C₆alkoxy and -NR²⁵R²⁶ (preferably, methoxy and N(Me)₂);
R²⁵ and R²⁶ are independently selected from C₁-C₆alkyl; or
R²⁵ and R²⁶ together with the nitrogen atom to which they are attached form a 4- to 6-membered heterocyclyl ring which optionally comprises one additional heteroatom individually selected from nitrogen, oxygen and sulfur;
and a compound of formula (VIII)

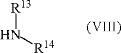

wherein R¹³ and R¹⁴ are as defined herein;
to produce a compound of formula (ll-a)

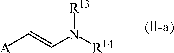

wherein A, R¹³ and R¹⁴ are as defined herein.

Scheme 1 below describes the reactions of the invention in more detail. The substituent definitions are as defined herein.

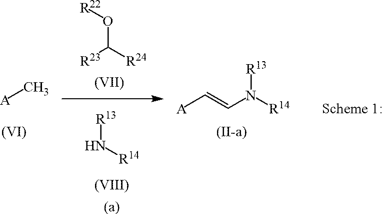

### Step (a) Formylation

Compounds of formula (ll-a) can be prepared by reacting a compound of formula (VI)

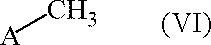

wherein A is as defined herein, with a compound of formula (VII)

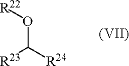

wherein R²², R²³ and R²⁴ are as defined herein;
and a compound of formula (VIII)

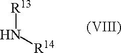

wherein R¹³ and R¹⁴ are as defined herein;
to produce a compound of formula (ll-a)

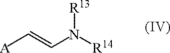

wherein A, R¹³ and R¹⁴ are as defined herein.

Typically the process described in step (a) is carried out in the presence of a catalytic amount of acid, or a catalytic mixture of acids, such as but not limited to, trifluoroacetic acid, acetic acid, benzoic acid, pivalic acid, propionic acid, butylated hydroxytoluene (BHT), 2,6-Di-tert-butylphenol, 2,4,6-Tri-tert-butylphenol, methanesulfonic acid, hydrochloric acid or sulfuric acid. Preferably, process step (a) is carried out in the presence of an acid with a non-alkylable anion, such as but not limited to butylated hydroxytoluene (BHT), 2,6-Di-tert-butylphenol or 2,4,6-Tri-tert-butylphenol.

The amount of acid is typically from 0.05 to 40 mol%(based on a compound of formula (VI)), preferably from 0.1 to 20 mol%.

The process described in step (a) may be carried out in the absence of a solvent, or in a solvent, or mixture of solvents, such as but not limited to, tetrahydrofuran, 2-methyltetrahydrofuran, diethylether, tert-butylmethylether, tert-amyl methyl ether, cyclopentyl methyl ether, dimethoxymethane, diethoxymethane, dipropoxy methane, 1,3-dioxolane, ethyl acetate, dimethyl carbonate, dichloromethane, dichloroethane, N,N-dimethylformamide, N,N-dimethylacetamide, N-methyl pyrrolidone (NMP), acetonitrile, propionitrile, butyronitrile, benzonitrile, toluene, 1,4-dioxane or sulfolane.

This step can be carried out at a temperature of from 0° C. to 230° C., preferably, from 150° C. to 230° C., more preferably from 180° C. to 220° C.

In another embodiment, this step can be carried out at a temperature of from 50° C. to 110° C.

The skilled person would appreciate that unreacted starting material, a compound of formula (VI), (VII) or (VIII) can be recovered and reused.

Preferably, this step is carried out in a closed vessel (for example but not limited to an autoclave).

Preferably, this step is carried out with the continuous removal (for example, but not limited, by fractional distillation under pressure) of by-products (for example methanol and/or ethanol). More preferably, wherein a compound of formula (VII) is trimethyl orthoformate or triethyl orthoformate the reaction is carried out with the continuous removal of methanol or ethanol.

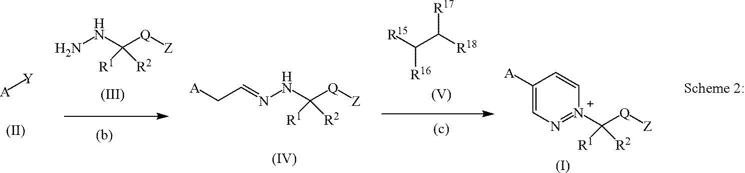

### Step (b) Hydrazone Formation

Compounds of formula (IV) can be produced by reacting a compound of formula (II)

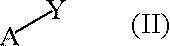

wherein A and Y are as defined herein,
with a compound of formula (III):

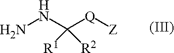

wherein R¹, R², Q and Z are as defined herein, to produce a compound of formula (IV),

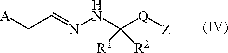

wherein A, Q, Z, R¹ and R² are as defined herein.

Typically the process described in step (b) can be carried out as a neat reaction mixture, however it may also be carried out in a solvent, or mixture of solvents, such as but not limited to, water, acetic acid, propionic acid, methanol, ethanol, propanol, isopropanol, tert-butanol, butanol, 3-methyl-1-butanol, tetrahydrofuran, 2-methyltetrahydrofuran, tert-butylmethylether, tert-amyl methyl ether, cyclopentyl methyl ether, dimethoxymethane, diethoxymethane, dipropoxy methane, 1,3-dioxolane, dimethyl carbonate, dichloromethane, dichloroethane, N,N-dimethylformamide, N,N-dimethylacetamide, N-methyl pyrrolidone (NMP), acetonitrile, propionitrile, butyronitrile, benzonitrile (or derivative thereof e.g 1,4-dicyanobenzene), 1,4-dioxane or sulfolane. Preferably process step (b) is carried out in water, acetonitrile, propionitrile or butyronitrile (or mixtures thereof).

Preferably, wherein Y is the group Y-I for a compound of formula (II), the process described in step (b) is carried out with the continuous removal (for example by distiallation) of the amine (HNR¹³R¹⁴) liberated.

Typically the process described in step (b) can be carried out in the presence of a Brönsted acid additive, or a mixture of Brönsted acid additives, such as but not limited to, trifluoroacetic acid, acetic acid, propionic acid, hydrochloric acid, sulfuric acid. Preferably, process step (b) is carried out in the presence of trifluoroacetic acid, hydrochloric acid, sulfuric acid or tetrafluoroboric acid.

The amount of acid additive is typically between 0.01 equivalent and 10 equivalents, preferably between 0.1 and 2 equivalents.

Typically the process described in step (b) can be carried out in a continuous fashion (for example, using a continuous distillation column).

Typically the process described in step (b) can be carried out at a temperature of from 0° C. to 120° C., preferably, from 10° C. to 50° C.

### Step (c) Cyclisation

The compound of formula (I) can be prepared by reacting a compound of formula (IV):

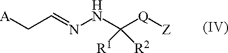

wherein A, Q, Z, R¹ and R² are as defined herein, with a compound of formula (V) or a salt or an N-oxide thereof;

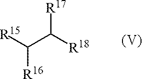

wherein each R^{15,} R¹⁶, R¹⁷ and R¹⁸ are as defined herein, to give a compound of formula (I)

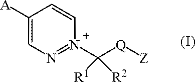

wherein A, Q, Z, R¹ and R² are as defined herein.

Typically process step (c) is carried out in the presence of a suitable additive enabling control of the pH of the reaction medium (preferably the pH of the reaction medium is from -0.5 to 6, more preferably from 0 to 6, even more preferably from 0 to 2.5), such as, but not limited to, morpholinium acetate, hydrochloric acid, trifluoroacetic acid, acetic acid, propionic acid, sulfuric acid, tartaric acid, oxalic acid, potassium hydrogenosulfate, sodium hydrogenosulfate, disodium phosphate or monosodium phosphate. Preferably, process step (c) is carried out in the presence of morpholinium acetate, trifluoroacetic acid, tartaric acid, oxalic acid, potassium hydrogenosulfate, hydrochloric acid or sulfuric acid. More preferably, process step (c) is carried out in the presence of hydrochloric acid, morpholinium acetate or tartaric acid.

Preferably, process step (c) is carried out in a suitable reaction medium at a pH of from -0.5 to 6. More preferably, process step (c) is carried out in a suitable reaction medium at a pH of from 0 to 6. Even more preferably, process step (c) is carried out in a suitable reaction medium at a pH of from 0 to 2.5.

The process described in step (c) can advantageously be carried out in the presence of a catalyst, preferably a lewis acid catalyst. More preferably, process step (c) is carried out in the presence of a Zirconium (Zr(IV)) or Scandium (Sc(lll)) salt, such as, but not limited to ZrCl₄, ZrOClz.8HzO, ScCl₃ or Sc(SO₃CF₃)₃. Even more preferably, process step (c) is carried out in the presence of a Zirconium (Zr(IV)) salt. Even more preferably still, process step (c) is carried out in the presence of ZrCl₄ or ZrOCl₂.8H₂O (preferably ZrOCl₂.8H₂O).

The amount of catalyst is typically from 0.05 to 40 mol%(based on a compound of formula (IV)), preferably from 0.1 to 20 mol%.

Typically the process described in step (c) is carried out in the absence of additional solvent (the skilled person would appreciate that where for example the compound of formula (V) is glyoxal (a compound of formula (Va), then this may be provided for example as a 40 wt % solution in water which may act as a solvent), or in the presence of a solvent, or mixture of solvents, such as but not limited to, water, acetic acid, propionic acid, methanol, ethanol, propanol, isopropanol, tert-butanol, butanol, 3-methyl-1-butanol, tetrahydrofuran, 2-methyltetrahydrofuran, diethylether, tert-butylmethylether, tert-amyl methyl ether, cyclopentyl methyl ether, dimethoxymethane, diethoxymethane, dipropoxy methane, 1,3-dioxolane, ethyl acetate, dimethyl carbonate, dichloromethane, dichloroethane, N,N-dimethylformamide, N,N-dimethylacetamide, N-methyl pyrrolidone (NMP), acetonitrile, propionitrile, butyronitrile, benzonitrile (or derivative thereof e.g 1,4-dicyanobenzene), 1,4-dioxane or sulfolane. Preferably the process described in step (c) is carried out in the absence of additional solvent, or in the presence of a solvent, or mixture of solvents, selected from the group consisting of water, methanol, ethanol, propanol, isopropanol, tert-butanol, butanol, acetonitrile, tetrahydrofuran and methyltetrahydrofuran.

In a preferred embodiment, this step is carried out in the presence of a Zirconium (Zr(IV)) salt and an alcohol solvent. More preferably, this step is carried out in the presence of ZrCl₄ or ZrOCl₂.8H₂O and methanol and/or ethanol.

The skilled person would appreciate that in process step (c), where for example the compound of formula (V) is glyoxal (a compound of formula (Va)), glyoxal can be efficiently removed from the reaction mixtures by several consecutive extractions (2-3) (or continuous extraction) with water-immiscible alcohols (via formation of hemiacetals). Examples of alcohols that can be used include but are not limited to Isoamylalkohol, 4-Methyl-2-pentanol, Hexanol, Octanol, 2-Phenylethanol and 3-Phenyl-1-propanol. Furthermore mixtures of an alcohol with a non-alcoholic solvent can also be used. The recovery of glyoxal from its hemiacetals is known.

Typically this step reaction can be carried out at a temperature of from -20° C. to 120° C., preferably, from -10° C. to 50° C.

The skilled person would appreciate that process steps (b) and (c) can be carried out in separate process steps, wherein the intermediate compounds can be isolated at each stage. Alternatively, the process steps (b) and (c) can be carried out in a one-pot procedure wherein the intermediate compounds produced are not isolated. Thus, it is possible for the process of the present invention to be conducted in a batch wise or continuous fashion.

The skilled person would appreciate that the temperature of the process according to the invention can vary in each of steps (a), (b) and (c). Furthermore, this variability in temperature may also reflect the choice of solvent used.

Preferably, the process of the present invention is carried out under an inert atmosphere, such as nitrogen or argon.

In a preferred embodiment of the invention the compound of formula (I) is further converted (for example via a hydrolysis, oxidation and/or a salt exchange as shown in scheme 3 below) to give an agronomically acceptable salt of formula (la) or a zwitterion of formula (Ib),

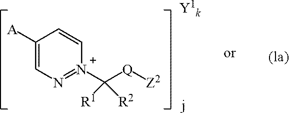

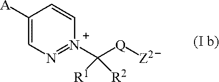

wherein Y¹ represents an agronomically acceptable anion and j and k represent integers that may be selected from 1, 2 or 3 (preferably, Y¹ is chloride (Cl⁻) or bromide (Br) and j and k are 1, more preferably, Y¹ is chloride (Cl⁻) or bromide (Br) and j and k are 1), and A, R′, R² and Q are as defined herein and Z² is —C(O)OH or —S(O)₂OH (the skilled person would appreciate that Z² represents —C(O)O⁻ or —S(O)₂O⁻).

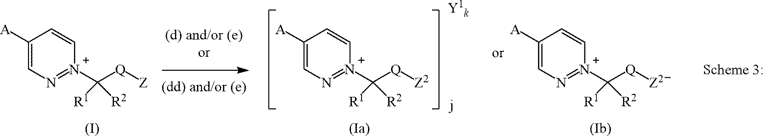

### Step (d) Hydrolysis

If required a hydrolysis can be performed using methods known to a person skilled in the art. The hydrolysis is typically performed using a suitable reagent, including, but not limited to aqueous sulfuric acid, concentrated hydrochloric acid or an acidic ion exchange resin.

Typically, the hydrolysis is carried out using aqueous hydrochloric acid (for example but not limited to, 32 wt% aq. HCl) or a mixture of HCl and an appropriate solvent, (such as but not limited to acetic acid, isobutyric acid or propionic acid), optionally in the presence of an additional suitable solvent (for example, but not limited to, water), at a suitable temperature from 0° C. to 120° C. (preferably, from 20° C. to 100° C.).

### Step (dd) Oxidation

Alternatively, where for example Z is —CH₂OH, an oxidation to the corresponding carboxylic acid wherein Z is —C(O)OH may be required instead of a hydrolysis. This oxidation can be performed using methods known to a person skilled in the art. One such method for example, is the oxidation of primary alcohols to corresponding carboxylic acids with a sodium hypochlorite (NaClO)/sodium chlorite (NaClO₂) system in the presence of 2,2,6,6-tetramethylpiperidine-1-oxyl (TEMPO) and related nitroxyl radicals as catalyst.

In this oxidation method examples of hypohalous acid salts which may be used include sodium hypobromite (NaBrO), sodium hypochlorite (NaClO) and potassium hypochlorite (KCIO). Examples of halous acid salts which may be used include sodium bromite (NaBrO₂), sodium chlorite (NaClO₂) and magnesium chlorite (Mg(ClO₂)₂). Examples of nitroxyl radicals which may be used include 2,2,6,6-tetramethylpiperidine-1-oxyl (TEMPO), 4-acetoamido-TEMPO, 4-carboxy-TEMPO, 4-amino-TEMPO, 4-phosphonoxy-TEMPO, 4-(2-bromoacetoamido)-TEMPO, 4-hydroxy-TEMPO, 4-oxy-TEMPO, 3-carboxyl-2,2,5,5-tetramethylpyrrolidin-1-oxyl, 3-carbamoyl-2,2,5,5-tetramethylpyrrolidin-1-oxyl and 3-carbamoyl-2,2,5,5-tetramethyl-3-pyrrolin-1-yloxyl.

The reaction typically requires a catalytic amount of sodium hypochlorite (eg, 5 - 10 mol%) for initiation of the reaction and at least a stoichiometric amount of sodium chlorite. The NaCIO/TEMPO system oxidizes the alcohol to the aldehyde and in situ the NaClO₂ oxidizes the aldehyde to carboxylic acid concomitantly generating 1 equivalent of NaClO, which is consumed in the oxidation of alcohol to aldehyde.

Other known methods for the oxidation of alcohols to aldehydes and aldehydes to carboxylic acids may be be used. For example, the direct oxidation of alcohol to carboxylic acid may be performed using hydrogen peroxide in the presence of a tungstate catalyst (eg, Na₂WO₄) - see, eg, Noyori R et al, Chem Commun (2003), 1977-1986.

### Step (e) Salt Exchange

If required the salt exchange of a compound of formula (I) to a compound of formula (la) can be performed using methods known to a person skilled in the art and refers to the process of converting one salt form of a compound into another (anion exchange), for example coverting a trifluoroacetate (CF₃CO₂⁻) salt to a chloride (Cl⁻) salt. The salt exchange is typically performed using an ion exchange resin or by salt metathesis. Salt metathesis reactions are dependent on the ions involved, for example a compound of formula (I) wherein the agronomically acceptable salt is a hydrogen sulfate anion (HSO₄⁻ ) may be switched to a compound of formula (la) wherein Y¹ is a chloride anion (Cl⁻) by treatment with an aqueous solution of barium chloride (BaCl₂) or calcium chloride (CaCl₂). Preferably, the salt exchange of a compound of formula (I) to a compound of formula (la) is performed with barium chloride.

In a preferred embodiment of the invention there is provided a process for the preparation of a compound of formula (I) or an agronomically acceptable salt or zwitterionic species thereof:

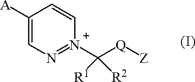

wherein
A is a 6-membered heteroaryl selected from the group consisting of formula A-la to A-IIIa (preferably A-la or A-IIIa) below

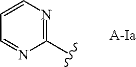

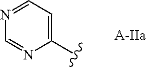

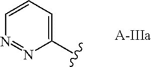

wherein the jagged line defines the point of attachment to the remaining part of a compound of formula (I); and
R¹ is hydrogen;
R² is hydrogen;
Q is (CR^{1a}R^{2b})_{m;}
m is 1;
each R^{1a} and R^{2b} are hydrogen;
Z is selected from the group consisting of —CN, —CH₂OH, —C(O)OR¹⁰, —S(O)₂OR¹⁰ and —CH═CH₂ (preferably —CN, —C(O)OR¹⁰, and —S(O)₂OR¹⁰, more preferably —CN and —C(O)OR¹⁰); and
R¹⁰ is selected from the group consisting of hydrogen and C₁-C₆alkyl (preferably, methyl, ethyl or tert-butyl);
said process comprising:
reacting a compound of formula (IV);

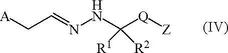

wherein A, Q, Z, R¹ and R² are as defined above;
with a compound selected from the group consisting of a compound of formula (Va), (Vc-ll), (Ve-I), (Vf-I) and (Vg-I) (preferably, a compound of formula (Va)),

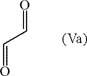

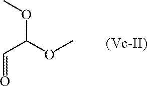

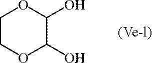

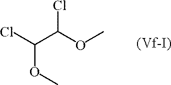

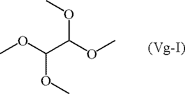

or a salt or an N-oxide thereof to give a compound of formula (I).

Preferably, there is provided a process for the preparation of a compound of formula (I) or an agronomically acceptable salt or zwitterionic species thereof:

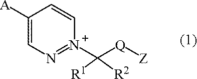

wherein
A is a 6-membered heteroaryl selected from the group consisting of formula A-la to A-IIIa (preferably A-la or A-IIIa) below

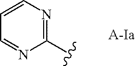

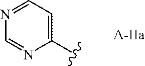

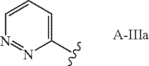

wherein the jagged line defines the point of attachment to the remaining part of a compound of formula (I); and
R¹ is hydrogen;
R² is hydrogen;
Q is (CR^{1a}R^{2b})_{m;}
m is 1;
each R^{1a} and R^{2b} are hydrogen;
Z is selected from the group consisting of —CN, —CH₂OH, —C(O)OR¹⁰, —S(O)₂OR¹⁰ and —CH═CH₂ (preferably —CN, —C(O)OR¹⁰, and —S(O)₂OR¹⁰, more preferably —CN and —C(O)OR¹⁰); and
R¹⁰ is selected from the group consisting of hydrogen and C₁-C₆alkyl (preferably, methyl, ethyl or tert-butyl);
said process comprising:
   reacting a compound of formula (IV); 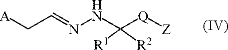
   wherein A, Q, Z, R¹ and R² are as defined above;
   with a compound selected from the group consisting of a compound of formula (Va), (Vc-II), (Ve-I), (Vf-I) and (Vg-I) (preferably a compound of formula (Va)), 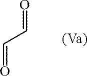 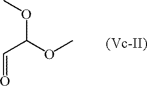 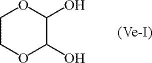 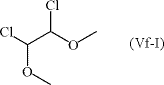 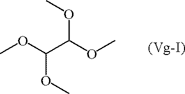
   or a salt or an N-oxide thereof, in a suitable reaction medium at a pH of from -0.5 to 6 (preferably, at a pH of from 0 to 2.5) to give a compound of formula (I).

In another preferred embodiment, there is provided a process for the preparation of a compound of formula (I) or an agronomically acceptable salt or zwitterionic species thereof:

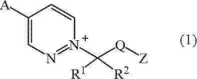

wherein
A is a 6-membered heteroaryl selected from the group consisting of formula A-la to A-IIIa (preferably A-la or A-IIIa) below

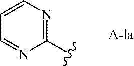

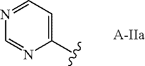

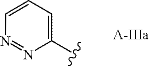

wherein the jagged line defines the point of attachment to the remaining part of a compound of formula (I); and
R¹ is hydrogen;
R² is hydrogen;
Q is (CR^{1a}R^{2b})ₘ;
m is 1;
each R^{1a} and R^{2b} are hydrogen;
Z is selected from the group consisting of —CN, —CH₂OH, —C(O)OR¹⁰, —S(O)₂OR¹⁰ and —CH═CH₂ (preferably —CN, —C(O)OR¹⁰, and —S(O)₂OR¹⁰, more preferably —CN and —C(O)OR¹⁰); and
R¹⁰ is selected from the group consisting of hydrogen and C₁-C₆alkyl (preferably, methyl, ethyl or tert-butyl);
said process comprising:
   reacting a compound of formula (IV); 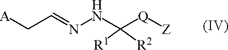
   wherein A, Q, Z, R¹ and R² are as defined above;
   with a compound selected from the group consisting of a compound of formula (Va), (Vc-II), (Ve-I), (Vf-I) and (Vg-I) (preferably, a compound of formula (Va)), 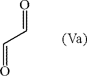 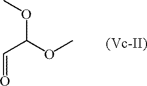 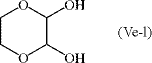 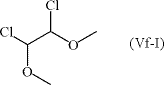 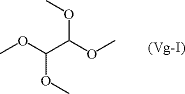
   or a salt or an N-oxide thereof;
   in a suitable reaction medium at a pH of from -0.5 to 6 (preferably, at a pH of from 0 to 2.5, more preferably at a pH of from 0 to 1.5) and in the presence of a Zirconium or Scandium salt (preferably, in the presence of ZrCl₄ or ZrOCl₂.8H₂O) to give a compound of formula (I).

### EXAMPLES

The following examples further illustrate, but do not limit, the invention. Those skilled in the art will promptly recognise appropriate variations from the procedures both as to reactants and as to reaction conditions and techniques.

The following abbreviations are used: s = singlet; br s = broad singlet; d = doublet; dd = double doublet; dt = double triplet; t = triplet, tt = triple triplet, q = quartet, quin = quintuplet, sept = septet; m = multiplet; GC = gas chromatography, RT = retention time, Tᵢ = internal temperature, MH⁺ = molecular mass of the molecular cation, M = molar, Q¹HNMR = quantitative ¹HNMR, RT = room temperature, UFLC = Ultra-fast liquid chromatography.

¹H NMR spectra are recorded at 400 MHz unless indicated otherwise and chemical shifts are recorded in ppm.

Some chemical yields have been calculated precisely using quantitative 1H NMR and 1,3,5-trimethoxybenzene or caffeine as an internal standard. Where the chemical yield is based on quantative 1H NMR the nature of any relevant counterion is assumed based on the reaction conditions used, however, the skilled person would appreciate that the crude reaction mixture may also include (but are not limited to) other counter ions such as chloride, bromide, iodide, fluoride, hydrogen sulfate, mesylate, oxalate, tartrate and trifluoroacetate.

### LCMS Methods

### Standard:

Spectra were recorded on a Mass Spectrometer from Waters (SQD, SQDII Single quadrupole mass spectrometer) equipped with an electrospray source (Polarity: positive and negative ions, Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 µm, 30 × 2.1 mm, Temp: 60° C., DAD Wavelength range (nm): 210 to 500, Solvent Gradient: A = water + 5% MeOH + 0.05% HCOOH, B= Acetonitrile + 0.05 % HCOOH, gradient: 10-100% B in 1.2 min; Flow (ml/min) 0.85

### Standard Long

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30 V, Extractor: 2.00 V, Source Temperature: 150° C., Desolvation Temperature: 350° C., Cone Gas Flow: 50 I/h, Desolvation Gas Flow: 650 I/h, Mass range: 100 to 900 Da) and an Acquity UPLC from Waters: Binary pump, heated column compartment, diode-array detector and ELSD detector. Column: Waters UPLC HSS T3, 1.8 µm, 30 × 2.1 mm, Temp: 60° C., DAD Wavelength range (nm): 210 to 500, Solvent Gradient: A = water + 5% MeOH + 0.05 % HCOOH, B= Acetonitrile + 0.05 % HCOOH, gradient: 10-100% B in 2.7 min; Flow (ml/min) 0.85

### Example 1: Preparation of Tert-Butyl 3-(4-Pyrimidin-2-Ylpyridazin-1-Ium-1-yl)Propanoate Trifluoroacetate Salt From Tert-Butyl 3-[2-(2-Pyrimidin-2-Ylethylidene)Hydrazinolpropanoate and Glyoxal

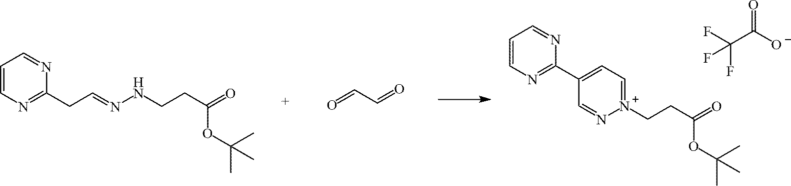

### Procedure:

Morpholinium acetate was prepared in situ by mixing morpholine (1 eq.) and acetic acid (1eq.)).

To a solution of morpholinium acetate (0.158 g, 1.07 mmol, 0.85 eq.), glyoxal (0.366 mL, 40% in H₂O) and trifluoroacetic acid (0.287 g, 2.52 mmol, 2 eq.) in dioxane (0.5 ml) was added a solution of tert-butyl 3-[2-(2-pyrimidin-2-ylethylidene)hydrazino]propanoate (0.333 g, 1.26 mmol) in dioxane (2.5 mL) via syringe pump over 3 h. The reaction mixture was stirred at room temperature for 15 h.

The mixture was then concentrated under reduced pressure. The chemical yield of tert-butyl 3-(4-pyrimidin-2-ylpyridazin-1-ium-1-yl)propanoate trifluoroacetate salt was determined using quantitative 1H NMR using 1,3,5-trimethoxybenzene as an internal standard to be 33%.

¹H NMR (400 MHz, MeOH-d4) δ ppm: 10.4(d, 1H), 10.04(d, 1H), 9.43(dd, 1H), 9.14(d, 2H), 7.72(t, 1H), 5.17(t, 2H), 3.24(t, 2H), 1.45(S, 9H)

### Example 2: Preparation of Tert-Butyl 3-(4-Pyrimidin-2-Ylpyridazin-1-Ium-1-yl)Propanoate Trifluoroacetate Salt From Tert-butyl 3-[2-(2-Pyrimidin-2-Ylethylidene)Hydrazino]Propanoate and 1,1,2,2-Tetramethoxyethane

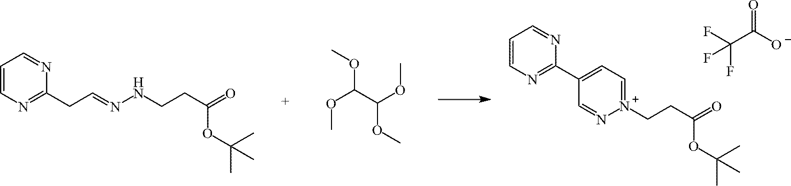

### Procedure

Morpholinium acetate was prepared in situ by mixing morpholine (1 eq.) and acetic acid (1eq.).

To a suspension of morpholinium acetate (0.158 g, 0.85 eq.) in dioxane (0.5 ml) were added in parallel a solution of tert-butyl 3-[2-(2-pyrimidin-2-ylethylidene)hydrazino]propanoate (0.333 g, 1.26 mmol, 1 eq.) in dioxane (1 mL) and a solution of 1,1,2,2-tetramethoxyethane (0.398 g, 2.00 eq.) and trifluoroacetic acid (0.287 g, 0.193 mL, 2.00 eq.) in dioxane (1 mL) using two syringe pumps over 2 h15 min. The chemical yield of tert-butyl 3-(4-pyrimidin-2-ylpyridazin-1-ium-1-yl)propanoate trifluoroacetate salt was determined using quantitative 1H NMR using 1,3,5-trimethoxybenzene (20 mg) as an internal standard to be 31%.

¹H NMR (400 MHz, MeOH-d4) δ ppm: 10.4(d, 1H), 10.04(d, 1H), 9.43(dd, 1H), 9.14(d, 2H), 7.72(t, 1H), 5.17(t, 2H), 3.24(t, 2H), 1.45(S, 9H)

### Example 3: Preparation of Tert-Butyl 3-(4-Pyrimidin-2-Ylpyridazin-1-Ium-1-yl)Propanoate Trifluoroacetate Salt From Tert-Butyl 3-[2-(2-Pyrimidin-2-Ylethylidene)Hydrazino]Propanoate and 2,2-Dimethoxyacetaldehyde

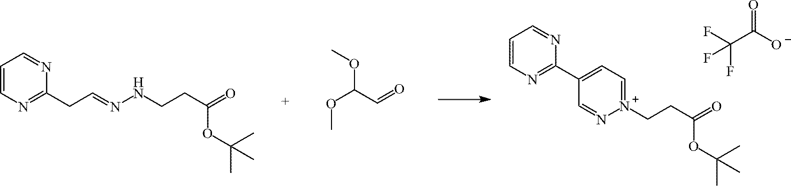

### Procedure

Morpholinium acetate was prepared in situ by mixing morpholine (1 eq.) and acetic acid (1eq.). tert-butyl 3-[2-(2-pyrimidin-2-ylethylidene)hydrazino]propanoate was prepared according to the procedure described below in Example 11. from tert-butyl 3-hydrazinopropanoate (0.134 g, 1.3 eq.) and (E)-N,N-dimethyl-2-pyrimidin-2-yl-ethenamine (0.1 g, 0.67 mmol, 1 eq.).

The resulting crude tert-butyl 3-[2-(2-pyrimidin-2-ylethylidene)hydrazino]propanoate 0.67 mmol, 1 eq.) was dissolved in dioxane (0.5 mL) and morpholinium acetate was added (0.093 g, 0.63 mmol, 0.94 eq.). The resulting suspension was stirred for 30 min at rt.

In a separate vial, 2,2-dimethoxyacetaldehyde (0.219 g, 0.19 mL, 60% w/w in H₂O) was mixed with trifluoroacetic acid (0.144 g, 0.096 mL, 2 eq.) and diluted with 1,4-dioxane (1.030 g, 1 mL). The resulting mixture of glyoxal-acetal/TFA dioxane solution was next added to the tert-butyl 3-[2-(2-pyrimidin-2-ylethylidene)hydrazino]propanoate solution in dioxane over 1h at RT.

The reaction mixture was further stirred at rt for 2 h, and then concentrated.1,3,5-trimethoxybenzene was added (21.5 mg) as an internal standard and the mixture was analyzed by quantitative 1H NMR in CD₃OD indicating the title compound had been formed in 4.3% yield.

¹H NMR (400 MHz, MeOH-d4) δ ppm: 10.4(d, 1H), 10.04(d, 1H), 9.43(dd, 1H), 9.14(d, 2H), 7.72(t, 1H), 5.17(t, 2H), 3.24(t, 2H), 1.45(S, 9H)

### Example 4: Preparation of 3-(4-Pyrimidin-2-Ylpyridazin-1-Ium-1-yl)Propanenitrile Trifluoroacetate Salt From 3-[2-(2-Pyrimidin-2-Ylethylidene)Hydrazino]Propanenitrile and Glyoxal

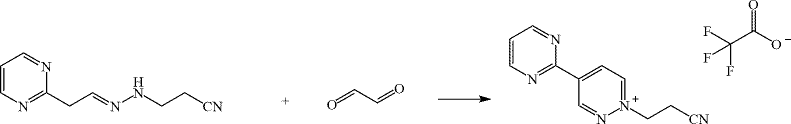

### Procedure:

Morpholinium acetate was prepared in situ by mixing morpholine (1 eq.) and acetic acid (1eq.). 3-[(2-(2-pyrimidin-2-ylethylidene)hydrazino]propanenitrile was prepared according to the procedure described in Example 15. in 70% yield

A vial was charged morpholinium acetate (0.277 g, 0.85 eq.), trifluoroacetic acid (0.340 mL, 2 eq.) and glyoxal (11.2 mL, 44 eq., 40 w/w% in H₂O) which was stirred to give a colorless homogeneous solution. 3-[(2-(2-pyrimidin-2-ylethylidene)hydrazino]propanenitrile (0.5 g, 2.2 mmol, 1 eq.) was then added as a solution in water (5 mL). After stirring for 22 h at room temperature the mixture was concentrated to give a yellow foam.

The chemical yield of tert-butyl 3-(4-pyrimidin-2-ylpyridazin-1-ium-1-yl)propanoate trifluoroacetate salt was determined using quantitative 1H NMR using caffeine as an internal standard to be 70%.

¹H NMR (400 MHz, D₂O) δ ppm: s(10.26, 1H), 9.93(d, 1H, 6.2 Hz), 9.29(dd, 1H, J=6.2, J=2.6 Hz), 9.03(d, 2H, 5.1 Hz), 7.68(t, 1H, 4.95 Hz), 5.23(t, 2H, J=6.4 Hz), 3.42(t, 2H, J=6.4 Hz)

### Example 5: Preparation of 3-(4-Pyrimidin-2-Ylpyridazin-1-Ium-1-yl)Propanenitrile Trifluoroacetate Salt From 3-[2-(2-Pyrimidin-2-Ylethylidene)Hydrazino]Propanenitrile and Glyoxal

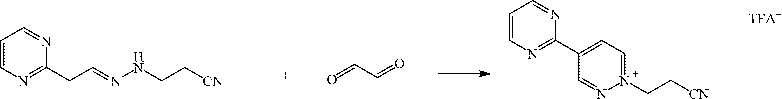

### Procedure

Morpholinium acetate was prepared in situ by mixing morpholine (1 eq.) and acetic acid (1eq.). 3-[(2-(2-pyrimidin-2-ylethylidene)hydrazino]propanenitrile was prepared according to the procedure described in Example 15 \. in 70% yield.

A vial was charged with trifluoroacetic acid (0.63 mL, 1.70 mmol, 2.00 eq, 2.67 M in H₂O), morpholinium acetate (106 mg, 0.72 mmol, 0.85 eq), glyoxal (618 mg, 4.25 mmol, 5.00 eq., 40% w/w in H₂O) and caffeine (0.85 mL, 0.085 mmol, 0.10 eq, 0.099 M in H₂O). 3-[(2-(2-pyrimidin-2-ylethylidene)hydrazino]propanenitrile (0.85 mmol, 0.33 mL, 2.54 M in THF) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 61% chemical yield.

¹H NMR (400 MHz, D₂O) δ ppm: 10.26(s, 1H), 9.93(d, 1H, 6.2 Hz), 9.29(dd, 1H, J=6.2, J=2.6 Hz), 9.03(d, 2H, 5.1 Hz), 7.68(t, 1H, 4.95 Hz), 5.23(t, 2H, J=6.4 Hz), 3.42(t, 2H, J=6.4 Hz)

### Example 6: Preparation of 3-(4-Pyrimidin-2-Ylpyridazin-1-Ium-1-yl)Propanenitrile Hydrogenosulfate Salt From 3-[2-(2-Pyrimidin-2-Ylethylidene)Hydrazino]Propanenitrile and Glyoxal

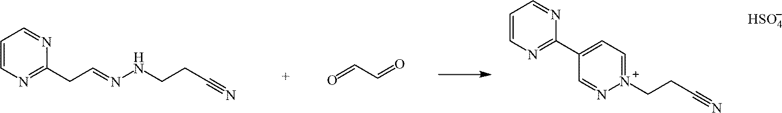

### Procedure:

Morpholinium acetate was prepared in situ by mixing morpholine (1 eq.) and acetic acid (1eq.). 3-[(2-(2-pyrimidin-2-ylethylidene)hydrazino]propanenitrile was prepared according to the procedure described in Example 15 \. in 70% yield.

A vial was charged with KHSO₄ (0.48 mL, 1.27 mmol, 1.5 eq, 2.67 M in H₂O), glyoxal (618 mg, 4.25 mmol, 5.00 eq., 40% w/w in H₂O) and caffeine (0.85 mL, 0.085 mmol, 0.10 eq, 0.099 M in H₂O). 3-[(2-(2-pyrimidin-2-ylethylidene)hydrazino]propanenitrile (0.85 mmol, 0.33 mL, 2.54 M in THF) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 54% chemical yield.

¹H NMR (400 MHz, D₂O) δ ppm: s(10.26, 1H), 9.93(d, 1H, 6.2 Hz), 9.29(dd, 1H, J=6.2, J=2.6 Hz), 9.03(d, 2H, 5.1 Hz), 7.68(t, 1H, 4.95 Hz), 5.23(t, 2H, J=6.4 Hz), 3.42(t, 2H, J=6.4 Hz)

### Example 7: Preparation of 3-(4-Pyrimidin-2-Ylpyridazin-1-Ium-1-yl)Propanenitrile Tartrate Salt From 3-[2-(2-Pyrimidin-2-Ylethylidene)Hydrazinolpropanenitrile and Glyoxal

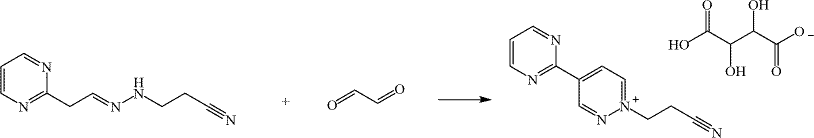

### Procedure:

3-[pyrimidin-2-ylethylidene)hydrazino]propanenitrile was prepared according to the procedure described in Example 15 \. in 70% yield.

A vial was charged with tartaric acid (382 mg, 2.55 mmol, 3 eq) and glyoxal (618 mg, 4.25 mmol, 5.00 eq., 40% w/w in H₂O) and caffeine (0.85 mL, 0.085 mmol, 0.10 eq, 0.099 M in H₂O). 3-[(2-(2-pyrimidin-2-ylethylidene)hydrazino]propanenitrile (0.85 mmol, 0.33 mL, 2.54 M in THF) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 44% chemical yield.

¹H NMR (400 MHz, D₂O) δ ppm: s(10.26, 1H), 9.93(d, 1H, 6.2 Hz), 9.29(dd, 1H, J=6.2, J=2.6 Hz), 9.03(d, 2H, 5.1 Hz), 7.68(t, 1H, 4.95 Hz), 5.23(t, 2H, J=6.4 Hz),

### Example 8: Preparation of 3-(4-pyrimidin-2-ylpyridazin-1-ium-1-yl)propanenitrile Trifluoroacetate Salt From 3-[2-(2-pyrimidin-2-ylethylidene)hydrazino]propanenitrile and 2,2-dimethoxyacetaldehyde

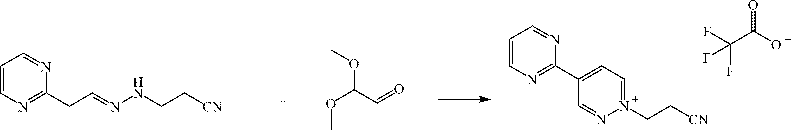

### Procedure

Morpholinium acetate was prepared in situ by mixing morpholine (1 eq.) and acetic acid (1eq.). 3-[(2-(2-pyrimidin-2-ylethylidene)hydrazino]propanenitrile was prepared according to the procedure described in Example 15 \. in 70% yield

A vial was charged with trifluoroacetic acid (0.63 mL, 1.70 mmol, 2.00 eq, 2.67 M in H₂O), morpholinium acetate (106 mg, 0.72 mmol, 0.85 eq), 2,2-dimethoxyacetaldehyde (736 mg, 4.25 mmol, 5.00 eq., 60% w/w in H₂O) and caffeine (0.85 mL, 0.085 mmol, 0.10 eq, 0.099 M in H₂O). 3-[(2-(2-pyrimidin-2-ylethylidene)hydrazino]propanenitrile (0.85 mmol, 0.33 mL, 2.54 M in THF) was next added. The vial was then sealed and stirred at room temperature for 24 h. After 24 h, 0.1 mL of the reaction mixture was sampled and diluted in D20 (0.5 ml) and analyzed by quantitative 1H NMR, indicating the title compound had been formed in 18% chemical yield.

¹H NMR (400 MHz, D₂O) δ ppm: (s, 10.26, 1H), 9.93(d, 1H, 6.2 Hz), 9.29(dd, 1H, J=6.2, J=2.6 Hz), 9.03(d, 2H, 5.1 Hz), 7.68(t, 1H, 4.95 Hz), 5.23(t, 2H, J=6.4 Hz), 3.42(t, 2H, J=6.4 Hz)

### Example 9: Preparation of 3-(4-Pyrimidin-2-Ylpyridazin-1-Ium-1-yl)Propanenitrile Trifluoroacetate Salt From 3-[2-(2-Pyrimidin-2-Ylethylidene)Hydrazino]Propanenitrile and 1,2-Dichloro-1,2-Dimethoxy-Ethane

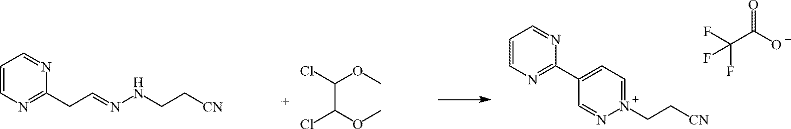

A vial was charged with 1,2-dichloro-1,2-dimethoxy-ethane (64 mg, 0.4 mmol, 2eq.), trifluoroacetic acid (0.80 mL, 0.4 mmol, 2.00 eq, 0.5 M in THF) and 1,3,5-trimethoxybenzene (10 mg, 0.059 mmol, 0.30 eq.). The mixture was stirred for 5 min. Acetic acid (0.34 mL, 0.17 mmol, 0.85 eq, 0.5 M in THF), morpholine (0.34 mL, 0.17 mmol, 0.85 eq, 0.5 M in THF) were added at rt and the mixture was stirred for 5 min. A THF solution of 3-[(2-(2-pyrimidin-2-ylethylidene)hydrazino]propanenitrile (0.20 mmol, 0.40 mL, 1.00 eq., 0.5 M in THF) was finally added. The vial was then sealed and stirred at room temperature for 1h. After 1 h, 0.1 mL of the reaction mixture was sampled and diluted in DMSO-d6 (0.5 ml) and analyzed by quantitative 1H NMR . Quantitative 1H NMR analysis (using 1,3,5-trimethoxybenezene as an internal standard), indicating the title compound had been formed in 23% chemical yield.

### Example 10: Preparation of 3-(4-Pyrimidin-2-Ylpyridazin-1-Ium-1-yl)Propanenitrile Trifluoroacetate Salt From 3-[2-(2-Pyrimidin-2-Ylethylidene)Hydrazino]Propanenitrile and 1,4-Dioxane-2,3-Diol

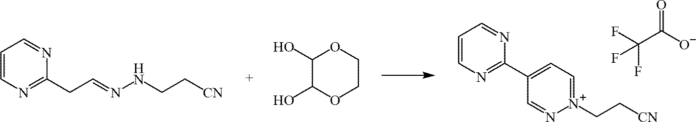

A vial was charged with 1,4-dioxane-2,3-diol (48 mg, 0.4 mmol, 2eq.), trifluoroacetic acid (0.80 mL, 0.4 mmol, 2.00 eq, 0.5 M in THF) and 1,3,5-trimethoxybenzene (10 mg, 0.059 mmol, 0.30 eq.). The mixture was stirred for 5 min. Acetic acid (0.34 mL, 0.17 mmol, 0.85 eq, 0.5 M in THF), morpholine (0.34 mL, 0.17 mmol, 0.85 eq, 0.5 M in THF) were added at rt and the mixture was stirred for 5 min. A THF solution of 3-[(2-(2-pyrimidin-2-ylethylidene)hydrazino]propanenitrile (0.20 mmol, 0.40 mL, 1.00 eq., 0.5 M in THF) was finally added. The vial was then sealed and stirred at room temperature for 1 h. After 1 h, 0.1 mL of the reaction mixture was sampled and diluted in DMSO-d6 (0.5 ml) and analyzed by quantitative 1H NMR. Quantitative 1H NMR analysis (using 1,3,5-trimethoxybenezene as an internal standard), indicating the title compound had been formed in 42% chemical yield

### Example 11: Preparation of Tert-Butyl 3-[2-(2-Pyrimidin-2-Ylethylidene)Hydrazino]Propanoate From Tert-Butyl 3-Hydrazinopropanoate and (E)-N,N-Dimethyl-2-Pyrimidin-2-yl-Ethenamine

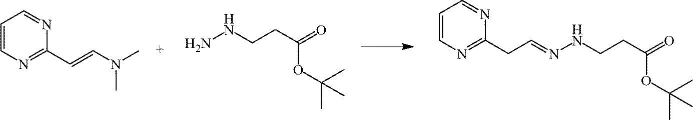

A vial was charged with N,N-dimethylenamine (1.5 g, 9.4 mmol, 1.00 eq.) and t-Butyl 3-hydrazino propanoate (1.77 g, 10.4 mmol, 1.10 eq.). The orange suspension was heated at 100° C. for 40 min under a flow of argon to help removing dimethylamine. The resulting mixture was then allowed to cool to room temperature. Quantitative 1H NMR analysis (using 1,3,5-trimethoxybenezene as an internal standard) of the crude mixture indicated the title compound had been formed in 76% yield as an E/Z mixture of hydrazone isomers.

¹H NMR (400 MHz, CDCl₃) δ ppm 8.70 (d, 2 H), 7.34 (t, 1 H), 7.19 (m, 1 H), 6.89 (t, 1 H), 3.92 (d, 2 H), 3.39 (t, 2 H), 2.54 (t, 2 H), 1.46 (s, 9 H)

### Example 12: Preparation of Tert-Butyl 3-[2-(2-Pyrimidin-2-Ylethylidene)Hydrazinolpropanoate From 2-[(2-Pyrrolidin-1-Ylvinyllpyrimidine

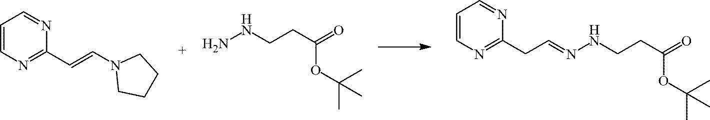

A vial was charged with 2-[(2-pyrrolidin-1-ylvinyl]pyrimidine (0.200 g, 1.2 mmol, 1.1 eq.) and tert-butyl 3-hydrazinopropanoate (0.256 g, 1.44 mmol, 1.1 eq.). The neat reaction mixture was warmed to 100° C. and set under 200 mbar for 1 h then under 1 mbar (to remove the pyrrolidine) for 1 h. The resulting mixture was then allowed to cool to room temperature. Quantitative 1H NMR analysis (using 1,3,5-trimethoxybenzene as an internal standard) of the crude mixture indicated the title compound had been formed in 50% chemical yield as an E/Z mixture of hydrazone isomers.

¹H NMR (400 MHz, CDCl₃) δ ppm 8.70 (d, 2 H), 7.34 (t, 1 H), 7.19 (m, 1 H), 6.89 (t, 1 H), 3.92 (d, 2 H), 3.39 (t, 2 H), 2.54 (t, 2 H), 1.46 (s, 9 H)

### Example 13: Preparation of Tert-Butyl 3-[2-(2-Pyrimidin-2-Ylethylidene)Hydrazino]Propanoate From 4-[2-Pyrimidin-2-Ylvinyllmorpholine

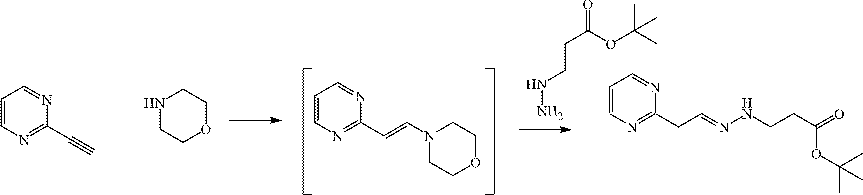

A vial was charged with 2-ethynylpyrimidine (490 mg, 4.60 mmol, 1.00 eq.) and THF (1.5 mL). Morpholine (615 mg, 7.00 mmol, 1.50 eq.) was then added via syringe. The reaction mixture was heated at 100° C. for 30 min. NMR analysis indicated ca. 90% conversion of the starting 2-alkynyl pyrimidine. Used as such in the subsequent step.

4-pyrimidin-2-ylvinyl]morpholine: ¹H NMR (400 MHz, CDCl₃) δ ppm 8.40 (d, 2 H), 7.65 (d, 2 H), 6.75 (t, 1 H), 5.5 (d, 1 H), 3.75 (m, 4 H), 3.25 (m, 2 H)

To the above THF solution of 4-[2-pyrimidin-2-ylvinyl]morpholine (4.60 mmol) was added a solution of tert-butyl 3-hydrazinopropanoate (0.930 g, 5.85 mmol, 1.26 eq.) in THF (0.5 mL) dropwise. The reaction mixture was heated at 100° C. for 60 min. The reaction mixture was concentrated in vacuo to give the crude product tert-butyl 3-[2-(2-pyrimidin-2-ylethylidene) hydrazino] propanoate (1.48 g) as an amber oil. The crude product was purified by flash chromatography on silica gel to give a yellow oil (0.517 g, 87% purity as determined by quant. 1H NMR using dimethylsulfone as internal standard, 51% yield).

tert-butyl 3-[2-(2-pyrimidin-2-ylethylidene)hydrazino]propanoate: ¹H NMR (400 MHz, CDCl₃) δ ppm 8.70 (d, 2 H), 7.34 (t, 1 H), 7.19 (m, 1 H), 6.89 (t, 1 H), 3.92 (d, 2 H), 3.39 (t, 2 H), 2.54 (t, 2 H), 1.46 (s, 9 H)

### Example 14: Preparation of Tert-Butyl 3-[2-(2-Pyrimidin-2-Ylethylidene)Hydrazino]Propanoate From 2-Ethynylpyrimidine and Tert-Butyl 3-Hydrazinopropanoate

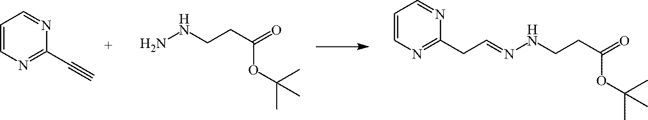

### Procedure

A vial was charged with 2-ethynylpyrimidine (1000 mg, 9.42 mmol, 1.00 eq.) and THF (1.0 mL/g) and then the resulting solution was heated to 50° C. under nitrogen atmosphere. A solution of tert-butyl 3-hydrazinopropanoate (1960 mg, 1.10 eq.) in THF (1.0 mL/g) was added. The reaction was then heated at 50° C. for 1 h. The solvent was then removed in vacuo to give the title compound as a brown oil (2400 mg, 63% purity as determined by quant. 1H NMR using mesitylene as internal standard, 61% yield).

### Example 15: Preparation of 3-[2-(2-Pyrimidin-2-Ylethylidene)Hydrazino]Propanenitrile From 2-[(2-Pyrrolidin-1-Ylvinyllpyrimidine

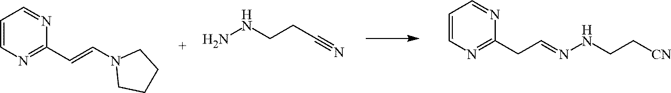

### Procedure

A flask was charged with 2-[(2-pyrrolidin-1-ylvinyl]pyrimidine (20 g, 114 mmol, 1.00 eq.) and THF (280 mL). To the above solution, 3-hydrazinopropanenitrile (20.4 g, 228 mmol, 2.00 eq.) was added in one portion at 20° C. under stirring. Trifluoroacetic acid (8.90 mL, 114 mmol, 1.00 eq.) was added dropwise at room temperature (maintaining temperature between 24° C.-26° C.). The reaction mixture was stirred at this temperature for 2 h. The reaction mixture was then concentrated under vacuo. The crude product was purified flash chromatography on silica gel to give (Isco Combiflash system on NP column) (Cyclohexane/ (EtOAc+EtOH 3:1) the title compound (19.5 g, 88% purity as determined by quant. NMR, 76% yield)

¹H NMR (400 MHz,CDCl₃) δ ppm: 8.72-8.69(m, 2H), 7.41(t, 0.6H, J=5.7), 7.22-7.18(m, 1H), 6.93(m, 0.4H,) 3.92-3.89(m, 2H), 3.54-3.41 (m, 2H), 2.68-2.65(m, 2H)

### Example 16: Preparation of 3-[2-(2-Pyrimidin-2-Ylethylidene)Hydrazino]Propanenitrile From 2-Ethynylpyrimidine

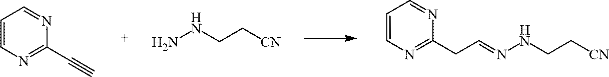

A vial was charged with 2-ethynylpyrimidine (1000 mg, 9.42 mmol, 1.00 eq.) and THF (1.0 mL/g) and then the resulting solution was heated to 50° C. under nitrogen atmosphere. A solution of 3-hydrazinopropanenitrile (900 mg, 1.1 eq.) in THF (1.0 mL/g) was added at 50° C. dropwise over 15 min then the reaction was stirred for 1h at 50° C. The solvent was then removed in vacuo to give the title compound as a brown oil (1650 mg, 68% purity as determined by quant. 1H NMR using mesitylene as internal standard, 61% yield. 50/50 mixture of E/Z hydrazone isomers).

### Example 17: Preparation of 3-(4-Pyridazin-3-Ylpyridazin-1-Ium-1-yl)Propanenitrile Trifluoroacetate Salt From 3-[2-(2-Pyridazin-3-Ylethylidene)Hydrazino]Propanenitrile and Glyoxal

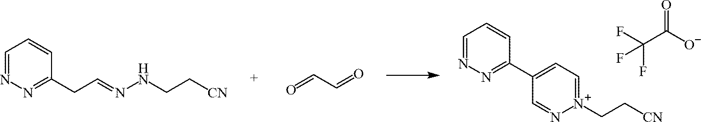

3-(2-pyridazin-3-ylethylidene)hydrazino]propanenitrile was prepared according to procedure described in Example 21.

A vial was charged with trifluoroacetic acid (0.63 mL, 1.70 mmol, 2.00 eq, 2.67 M in H₂O), morpholinium acetate (106 mg, 0.72 mmol, 0.85 eq), glyoxal (618 mg, 4.25 mmol, 5.00 eq., 40% w/w in H₂O) and caffeine (0.85 mL, 0.085 mmol, 0.10 eq, 0.099 M in H₂O). 3-[2-(2-pyrimidin-2-ylethylidene)hydrazino]propanenitrile ( 187 mg, 0.85 mmol, 86% purity) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 60% chemical yield.

1H NMR (400 MHz, D₂O) δ ppm: 10.18(s, 1H), 9.88(d, 1H, J=6.2 Hz), 9.32(d, 1H, 5.1 Hz), 9.16(dd, 1H, J=6.4, J=2.4 Hz), 8.52(d, 1H, J=8.8 Hz), 8.01-7.98,(m, 1H), 5.19(t, 2H, J=6.2 Hz), 3.37(t, 2H, 6.2 Hz)

### Example 18: Preparation of 3-(4-Pyridazin-3-Ylpyridazin-1-Ium-1-yl)Propanenitrile Trifluoroacetate Salt From 3-[2-(2-Pyridazin-3-Ylethylidene)Hydrazino]Propanenitrile and 2,2-Dimethoxyacetaldehyde

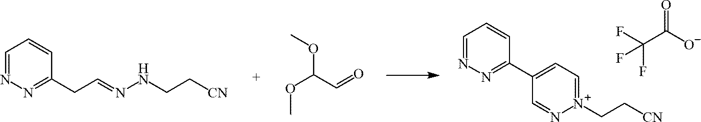

3-(2-pyridazin-3-ylethylidene)hydrazino]propanenitrile was prepared according to procedure described in Example 21.

A vial was charged with trifluoroacetic acid (0.63 mL, 1.70 mmol, 2.00 eq, 2.67 M in H₂O), morpholinium acetate (106 mg, 0.72 mmol, 0.85 eq), 2,2-dimethoxyacetaldehyde (736 mg, 4.25 mmol, 5.00 eq., 40% w/w in H₂O) and caffeine (0.85 mL, 0.085 mmol, 0.10 eq, 0.099 M in H₂O). 3-[2-(2-pyrimidin-2-ylethylidene)hydrazino]propanenitrile ( 187 mg, 0.85 mmol, 86% purity) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 12% chemical yield.

1H NMR (400 MHz, D₂O) δ ppm: 10.18(s, 1H), 9.88(d, 1H, J=6.2 Hz), 9.32(d, 1H, 5.1 Hz), 9.16(dd, 1H, J=6.4, J=2.4 Hz), 8.52(d, 1H, J=8.8 Hz), 8.01-7.98,(m, 1H), 5.19(t, 2H, J=6.2 Hz), 3.37(t, 2H, 6.2 Hz)

### Example 19: Preparation of 3-(4-Pyrimidin-2-Ylpyridazin-1-Ium-1-yl)Propanenitrile Trifluoroacetate Salt From 3-[2-(2-Pyrimidin-2-Ylethylidene)Hydrazino]Propanenitrile and 1,4-Dioxane-2,3-Diol

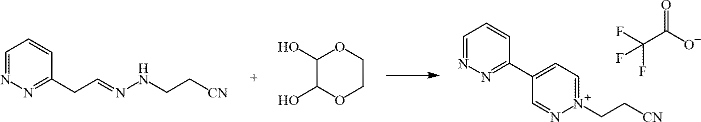

3-(2-pyridazin-3-ylethylidene)hydrazino]propanenitrile was prepared according to procedure described in Example 21.

A vial was charged with trifluoroacetic acid (0.63 mL, 1.70 mmol, 2.00 eq, 2.67 M in H₂O), morpholinium acetate (106 mg, 0.72 mmol, 0.85 eq), 1,4-dioxane-2,3-diol (510 mg, 4.25 mmol, 5.00 eq., 40% w/w in H₂O) and caffeine (0.85 mL, 0.085 mmol, 0.10 eq, 0.099 M in H₂O). 3-[2-(2-pyrimidin-2-ylethylidene)hydrazino]propanenitrile ( 187 mg, 0.85 mmol, 86% purity) was next added. The vial was then sealed and stirred at room temperature for 24 h. After 24 h, 0.1 mL of the reaction mixture was sampled and diluted in D₂O (0.5 ml) and analyzed by quantitative 1H NMR, indicating the title compound had been formed in 56% chemical yield.

1H NMR (400 MHz, D₂O) δ ppm: 10.18(s, 1H), 9.88(d, 2H, J=6.2 Hz), 9.32(d, 1H, 5.1 Hz, 9.16(dd, 1H, J=6.4, J=2.4 Hz), 8.52(d, 1H, J=8.8 Hz), 8.01-7.98,(m, 1H), 5.19(t, 2H, J=6.2 Hz), 3.37(t, 6.2 Hz)

### Example 20: Preparation of Tert-Butyl 3-[2-(2-Pyridazin-3-Ylethylidene)Hydrazino]Propanoate From 3-[2-Pyrrolidin-1-Ylvinyllpyridazine

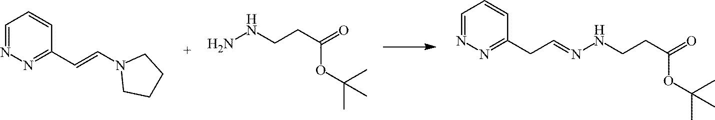

A vial was charged was charged with 3-[2-pyrrolidin-1-ylvinyl]pyridazine (5.0 g, 2.7 mmol, 1.0 eq.) and t-Butyl 3-hydrazino propanoate (6.09 g g, 35.7 mmol, 1.3 eq.). The neat reaction mixture was warmed to 100° C. and set under a flow of argon for 2 h. The reaction mixture was next put under high vacuum (1 mbar) to remove the pyrrolidine. The desired compound was obtained as a mixture of E/Z compound in 79% of yield (purity=68%, quantitative 1H NMR, Trimethoxybenzene as standard)

Work up: no work up. Used as such in the same time.

NMR data: 1H NMR (400 MHz, METHANOL-d4) δ ppm: 9.11-9.07(m, 1H), 7.70-7.68(m, 2H), 7.28(t, J=5.3 Hz, 0.75H), 6.72(t, J=5.2 Hz, 0.25H), 3.88-3.85(m, 2H), 3.42(t, J=6.8 Hz, 0.5H), 3.29(t, J=6.6 Hz, 1.5H), 2.53-2.45(m, 2H), 1.46(m, 9H)

### Example 21: Preparation of 3-[2-(2-Pyridazin-3-Ylethylidene)Hydrazino]Propanenitrile From 3-[2-Pyrrolidin-1-Ylvinyllpyridazine

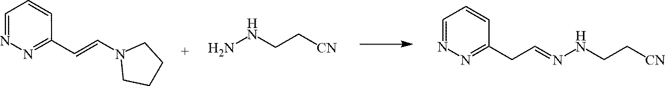

### Procedure 1

3-hydrazinopropanenitrile (5.28 g, 1.15 equiv., 62 mmol) was dissolved in water (10 mL). H₂SO₄ (2.88 g, 0.53 eq., 28.73 mmol) was then added dropwise to control the strong exotherm. Isobutyronitrile (50 mL, 10 eq., 550 mmol) was next added followed by 3-[2-pyrrolidin-1-ylvinyl]pyridazine (10.0 g, 54 mmol, 1.00 eq. 95% purity). The reaction mixture was stirred at rt for 1 hour. Reaction control (1H NMR) indicated ~92% conversion.

The reaction was then poured into a separating funnel and phases were separated. The organic phase was evaporated in vacuo (first at 90 mbar then at 25 mbar for 45 minutes). The title product was obtained as a deep red brown oil. (8.2 g, 86% purity as determined by quant. 1H NMR using trimethoxybenzene as an internal standard, 68% yield)

NMR data (mixture of isomers): 1H NMR (400 MHz, DMSO-d6) δ ppm: 9.14-9.09 (m, 1H), 7.65-7.56(m, 2H), 7.25(t, J=5.5 Hz, 0.81H), 6.82(t, J=5.7 Hz, 0.1H), 3.92(d, J=5.5 Hz, 1.5H), 3.81-3.79(m, 2H), 3.25-3.29(m, 0.5H), 3.22-3.18 (m, 1.5 H), 2.70 (t, J=6.6 Hz, 0.5H), 2.63(t, J=6.6 Hz, 1.5H)

### Procedure 2

3-pyrrolidin-1-ylvinyl]pyridazine (10.0 g, 54 mmol, 1.00 eq. 95% purity) was dissolved in THF (140 mL) and 3-hydrazinopropanenitrile (10.23 g, 2.00 equiv., 114 mmol) was added in one portion at room temperature. To this solution was added dropwise via a dropping funnel trifluoroacetic acid (4.44 mL, 54 mmol, 1.00 eq.) while maintaining the internal temperature below 26° C. The reaction mixture was then stirred at rt for 1 hour. The reaction mixture was concentrated in vacuo to give the title product as yellow oil as E/Zmixture( 75:25, unassigned) (27.0 g, 36% purity as determined by quant. 1H NMR using trimethoxy benzene as an internal standard, 93% yield)

NMR data: 1H NMR (400 MHz, CDCl3) δ ppm: 9.12-9.10( m, 1H), 7.50-7.41(m, 2H), 7.35(t, J=5.5 Hz, 0.75H), 6.83(t, J=5.7 Hz, 0.25H), 3.92(d, J=5.5 Hz, 1.5H), 3.85(d, J=5.9 Hz, 0.25H), 3.53-3.40(m, 2H), 2.67-2.63(m, 2H)

### Procedure 3

A 500 mL 3-Neck Round-bottom flask equipped with a 15 cm Vigreux column, a thermometer and a magnetic stirring bar was charged with 3-[2-pyrrolidin-1-ylvinyl]pyridazine (50.0 g, 0.283 mol), 3-hydrazinopropanenitrile (26.8 g, 0.309 mol) and 3-Methyl-1-butanol (102 g). Volatiles (a mixture of pyrrolidine and 3-methyl-1-butanol) were slowly distilled off at 40-45° C. (internal temperature) under vacuum (10-14 mbar) during 5 h. To the remaining residue more 3-Methyl-1-butanol (20 g) was added and the distillation was continued for 1 h under the same conditions. The conversion was monitored by NMR. The remaining residue was dried under full vacuum at 60° C. (jacket temperature)

The title compound (final residue) was obtained in 94 % yield as a brown oil (58.8 g, mixture of E/Z isomers, 86.2% purity as determined by quantitative 1H NMR in DMSO-d6 using Diethylene glycol diethyl ether as standard)

NMR data (mixture of isomers): 1H NMR (400 MHz, DMSO-d6) δ ppm: 9.13-9.09 (m, 1H), 7.65-7.55 (m, 2H), 7.25 (t, J=5.5 Hz, 0.75H), 6.83 (m, 1H), 6.62 (td, J=5.1 Hz, J=1.3 Hz, 0.25H), 3.81-3.78 (m, 2H), 3.35-3.30 (m, 0.5H), 3.23-3.18 (m, 1.5 H), 2.69 (t, J=6.6 Hz, 0.5H), 2.63 (t, J=6.6 Hz, 1.5H)

### Example 22: Preparation 3-Hydrazinopropanoic Acid (Aqueous Solution of Its Sodium Salt)

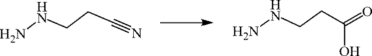 [SMILES: N#CCCNN ; NNCCC(=O)O]

A 20 mL vial was charged with 3-hydrazinopropanenitrile (2.00 g, 22.8 mmol) and 30% aqueous sodium hydroxide solution (3.65 g, 27.4 mmol, 1.2 eq.). The mixture was slowly heated to 70° C. When the gas evolution ceased, the mixture was heated to 110° C. and stirred at this temperature for 1 h. After cooling to room temperature, the pH of the reaction mixture was adjusted to 10.0 with 20% sulfuric acid (2.19 g, 0.20 eq). The resulting solution was concentrated by rotary evaporation to result in the title compound as a turbid pale yellow viscous oil in 77% yield. (purity=47% as for free acid, quantitative 1H NMR in D20 with Diethylene glycol diethyl ether as standard; contains sodium sulfate and the residual amount of water).

NMR data: 1H NMR (400 MHz, D₂O) δ ppm: 2.98 (t, J=7.2 Hz, 2H), 2.40 (t, J=7.2 Hz, 2H). H₂N-NH- protons are not visible because of H/D exchange.

### Example 23: Preparation 3-[2-(2-Pyridazin-3-Ylethylidene)Hydrazino]Propanoic Acid (Sodium Salt)

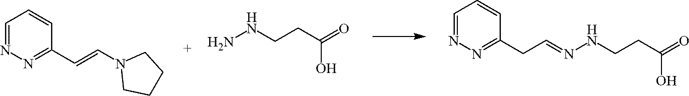

A small round-bottom flask was charged with 3-[2-pyrrolidin-1-ylvinyl]pyridazine (0.241 g, 1.36 mmol), 3-hydrazinopropanoic acid (sodium salt) from the previous example (0.346 g, 1.56 mmol, 1.15 eq.) and water (2 mL). The resulting solution was slowly concentrated by rotary evaporation (30° C., 30 mbar). Water (2 mL) was added to the residue and the resulting solution was concentrated again. This procedure was repeated 3 more times. The desired compound (final residue) was obtained as a mixture of E/Z isomers in 88% yield (purity=40.3% as for free acid, quantitative 1H NMR in D₂O with Diethylene glycol diethyl ether as standard)

NMR data: 1H NMR (400 MHz, D₂O) δ ppm: 9.13-9.09 (m, 1H), 7.82-7.74 (m, 2H), 7.48 and 6.96 (m, together 1H), 3.94 and 3.92 (d, J=5.5 Hz, <2H due to fast H/D exchange at this position), 3.40 and 3.27 (t, J=7.0 Hz, together 2H), 2.47 and 2.42 (t, J=7.0 Hz, together 2H). NH proton is not visible because of H/D exchange.

### Example 24: Preparation of 3-(4-Pyridazin-3-Ylpyridazin-1-Ium-1-yl)Propanoic Acid

### Hydrogenosulfate Salt

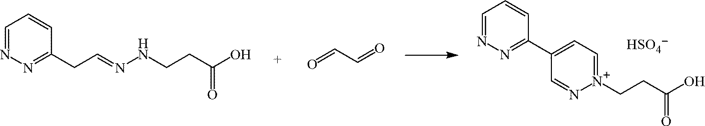

To a round bottom flask containing 3-[2-(2-pyridazin-3-ylethylidene)hydrazino]propanoic acid (0.607 g, 1.18 mmol, purity 40.3%, sodium salt) was added a mixture of KHSO₄ (0.404 g, 2.97 mmol, 2.5 eq.) and glyoxal (738 mg, 5.09 mmol, 4.3 eq., 40% w/w in H₂O) in one portion. The mixture was stirred at 40° C. for 2 h. The reaction mixture was sampled and analyzed by quantitative 1H NMR (in D20 with Diethylene glycol diethyl ether as standard), indicating the title compound had been formed in 17% chemical yield.

NMR data: ¹H NMR (400 MHz, D₂O) δ ppm: 10.23 (d, J=2.6 Hz, 1H), 10.00 (d, J=6.3 Hz, 1H), 9.45 (dd, J=5.1 Hz, 1.5 Hz, 1H), 9.23 (dd, J=6.3 Hz, 2.6 Hz, 1H), 8.63 (dd, J=8.7 Hz, 1.5 Hz, 1H), 8.12 (dd, J=8.7 Hz, 5.1 Hz, 1H), 5.24 (t, J=6.2 Hz, 2H), 3.34 (t, J=6.2 Hz, 2H).

### Example 25: Preparation of 2-[(2-Pyrrolidin-1-Ylvinyl]Pyrimidine

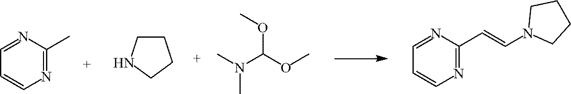

A mixture of 2-methyl-pyrimidine (10 g, 0.1063 mol), pyrrolidine (15.2 g, 0.2125 mol) and N,N-dimethylformamide dimethyl acetal (26.1 g, 0.2125 mol) was heated at 87° C. (internal temperature) for 15 h. After cooling down to room temperature, the mixture was concentrated under vacuum to give a yellowish solid. 300 ml of tButyl-methyl-ether were added to this solid, and it was dissolved at reflux. The solution was then cooled down to 0° C., stirred for 20 minutes, the solid was filtered, washed once with cold tButyl-methyl-ether, collected and dried under high vacuum. 12.3 g of 2-[(E)-2-pyrrolidin-1-ylvinyl] pyrimidine, a white solid, pure at 97%w/w as measured by Quantitative NMR was obtained. The filtrate was concentrated under vacuum and 200ml of tButyl-methyl-ether was added. After full dissolution was achieved at reflux, the solution was then cooled down to 0° C., stirred for 20 minutes, the solid was filtered, washed once with cold tButyl-methyl-ether, collected and dried under high vacuum. 4.7 g of 2-[2-pyrrolidin-1-ylvinyl]pyrimidine, a white solid, pure at 94%w/w as measured by Quantitative NMR was obtained. The two batches were combined to deliver 17 g of the title compound, pure at 96%w/w (84.1 % yield).

1H NMR (400 MHz, CDCl3) δ ppm 1.85 - 2.05 (m, 4 H) 3.28 - 3.44 (m, 4 H) 5.25 (d, 1 H) 6.67 (t, 1 H) 7.99 (d, 1 H) 8.38 (d, 2H).

### Example 26: Preparation of 4-[2-Pyrimidin-2-Ylvinyl]Morpholine

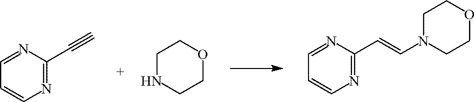

A mixture of 2-ethynylpyrimidine (0.25 g, 2.33 mmol) and morpholine (0.43 g, 4.89 mmol) was heated at 100° C. for 20 minutes. The mixture was then cooled down to room temperature and concentrated under vacuum. The crude title compound was obtained as an orange oil which solidified on standing (0.553 g) with a purity of 75%w/w as measured by Quantitative NMR. Most of the contaminant was residual morpholine.

1H NMR (400 MHz, CDCl3) δ ppm 3.23 - 3.33 (m, 4 H) 3.74 - 3.79 (m, 4 H) 5.49 (d, J=13.57 Hz, 1 H) 6.78 (t, J=4.95 Hz, 1 H) 7.66 (d, J=13.20 Hz, 1 H) 8.44 (d, J=4.77 Hz, 2 H)

### Example 27: Preparation of 2-[2-(1-Piperidyl)Vinyl]Pyrimidine

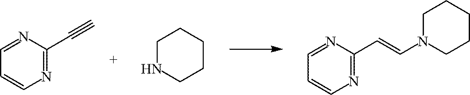

A mixture of 2-ethynylpyrimidine (0.25 g, 2.33 mmol) and piperidine (4.89 mmol) was heated at 100° C. for 20 minutes. The mixture was then cooled down to room temperature and concentrated under vacuum. The crude title compound was obtained.

1H-NMR (400 MHz, THF-d8) δ ppm 8.37 (d, J=4.77 Hz, 2 H), 7.76 (d, J=13.57 Hz, 1 H), 6.70 (t, J=4.77 Hz, 1 H), 5.43 (d, J=13.20 Hz, 1 H), 3.19 - 3.30 (m, 4 H), 1.56 - 1.67 (m, 6 H)

### Example 28: Preparation of 3-[2-Pyrrolidin-1-Ylvinyl]Pyridazine From 3-Methylpyridazine, Triethyl Orthoformate and Pyrrolidine in the Presence of 2,6-Di-Tert-Butyl-4-Methylphenol as Catalyst

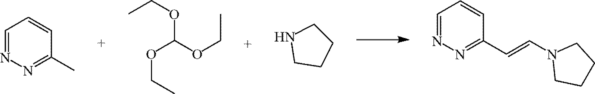

A 10 mL-microwave vial was charged with 3-methlypyridazine (0.55 g, 5.7 mmol), pyrrolidine (0.51 g, 7.2 mmol), triethyl orthoformate (1.14 g, 7.6 mmol) and 2,6-Di-tert-butyl-4-methylphenol (22 mg, 0.10 mmol, 2 mol%). The mixture was heated under stirring in a microwave reactor at 190° C. for 12 h. After cooling to room temperature, the reaction mixture was weighted, sampled and analyzed by quantitative 1H NMR (in DMSO-d6 with 1,3,5-trimethoxybenzene as standard), indicating the title compound had been formed in 55% chemical yield or 95% chemical yield based on converted starting material (58% conversion).

NMR data: 1H NMR (400 MHz, CDCl3) δ ppm: 8.60 (dd, J=4.6 Hz, 1.7 Hz, 1H), 7.80 (d, J=13.5 Hz, 1H), 7.31-7.23 (m, 2H), 5.10 (d, J=13.5 Hz, 1H), 3.28 (m, 4H), 1.88 (m, 4H).

### Example 29: Preparation of 3-[2-Pyrrolidin-1-Ylvinyl]Pyridazine From 3-Methylpyridazine, Trimethyl Orthoformate and Pyrrolidine in the Presence of 2,6-Di-Tert-Butyl-4-Methylphenol As Catalyst

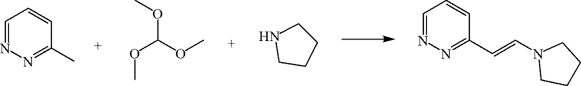

A 10 mL- microwave vial was charge with 3-methlypyridazine (0.97 g, 10 mmol), pyrrolidine (0.85 g, 12 mmol), trimethyl orthoformate (1.61 g, 15 mmol) and 2,6-Di-tert-butyl-4-methylphenol (45 mg, 0.20 mmol, 2 mol%). The mixture was heated under stirring in a microwave reactor at 200° C. for 9 h. After cooling to room temperature, the reaction mixture was weighted, sampled and analyzed by quantitative 1H NMR (in DMSO-d6 with 1,3,5-trimethoxybenzene as standard), indicating the title compound had been formed in 33% chemical yield or quantitative chemical yield based on converted starting material (33% conversion).

NMR data: 1H NMR (400 MHz, CDCl3) δ ppm: 8.60 (dd, J=4.6 Hz, 1.7 Hz, 1H), 7.80 (d, J=13.5 Hz, 1H), 7.31-7.23 (m, 2H), 5.10 (d, J=13.5 Hz, 1H), 3.28 (m, 4H), 1.88 (m, 4H).

### Example 30: Preparation of 2-[2-Pyrrolidin-1-Ylvinyl]Pyrimidine From 2-Methylpyrimidine, Triethyl Orthoformate and Pyrrolidine in the Presence of 2,6-Di-Tert-Butyl-4-Methylphenol as Catalyst

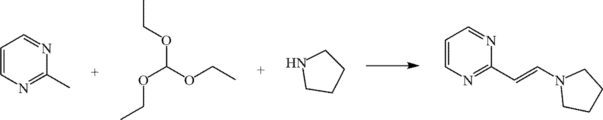

A 10 mL- microwave vial was charge with 2-methylpyrimidine (0.94 g, 10 mmol), pyrrolidine (0.85 g, 12 mmol), triethyl orthoformate (2.25 g, 15 mmol) and 2,6-Di-tert-butyl-4-methylphenol (45 mg, 0.20 mmol, 2 mol%). The mixture was heated under stirring in a microwave reactor at 220° C. for 4 h. After cooling to room temperature, the reaction mixture was weighted, sampled and analyzed by quantitative 1H NMR (in DMSO-d6 with 1,3,5-trimethoxybenzene as standard), indicating the title compound had been formed in 39% chemical yield or quantitative chemical yield based on converted starting material (39% conversion).

NMR data: 1H NMR (400 MHz, CDCl3) δ ppm: 8.34 (d, J=4.8 Hz, 2H), 7.91 (d, J=13.1 Hz, 1H), 6.75 (t, J=4.8 Hz, 1H), 5.04 (d, J=13.1 Hz, 1H), 3.28 (m, 4H), 1.88 (m, 4H).

### Example 31: Preparation of 3-(4-Pyridazin-3-Ylpyridazin-1-Ium-1-yl)Propanenitrile Chloride Salt From 3-[2-(2-Pyridazin-3-Ylethylidene)Hydrazino]Propanenitrile and Glyoxal in the Presence of ZrOCl₂*8H₂O

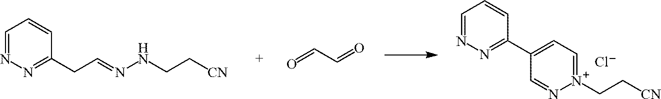

### Glyoxal (38.4 G, 0.265 Mol, 2.0 Eq., 40% W/w in H₂O) and Hydrochloric Acid (18.1 g, 0.159, 1.2 eq. 32% w/w in H₂O) Were Mixed (Solution 1)

### 3-(2-Pyridazin-3-Ylethylidene)Hydrazino]Propanenitrile (29.0 G, 0.132 Mol, 86.2%) and Methanol (17.0 g, 4.0 eq.) Were Mixed (Solution 2)

Solution 1 (11.3 g, 20% of the total amount) and Zirconium(IV) oxychloride octahydrate (4.35 g, 13 mmol, 10 mol%) were charged in a flask and the resulting solution was cooled to 0° C. Methanol (4.24 g, 1 eq.) was added and the mixture was stirred at 0-5° C. for 10 min

Solution 1 and solution 2 were dosed in parallel within 1 h while keeping the temperature at 0-5° C. After the end of addition, the reaction mixture was stirred for 1 h at 0-5° C. then for 2 h at room temperature. Water (33 ml) was added and methanol was distilled off in vacuum (100 → 25 mbar) at 45° C. (external temperature). 3-Methyl-1-butanol (67 mL) was added and the mixture was stirred at 45° C. for 1h. The phases were separated, and the aqueous phase was stirred again with fresh 3-Methyl-1-Butanol (67 mL) at 45° C. for 1 h. The phases were separated and the aqueous phase was concentrated to dryness by rotary evaporation to result in the title compound as an black-brown amorphous (glass-like) solid in 79% yield (42.9 g, purity=60%, quantitative 1H NMR in D₂O with 1-Methyl-2-pyridone as standard).

NMR data: ¹H NMR (400 MHz, D₂O) δ ppm: 10.26 (d, J=2.6 Hz, 1H), 10.03 (d, J=6.3 Hz, 1H), 9.38 (dd, J=5.1 Hz, 1.5 Hz, 1H), 9.27 (dd, J=6.3 Hz, 2.6 Hz, 1H), 8.60 (dd, J=8.7 Hz, 1.5 Hz, 1H), 8.07 (dd, J=8.7 Hz, 5.1 Hz, 1H), 5.32 (t, J=6.2 Hz, 2H), 3.49 (t, J=6.2 Hz, 2H).

### Example 32: Preparation of 3-(4-Pyridazin-3-Ylpyridazin-1-Ium-1-yl)Propanenitrile Chloride Salt From 3-[2-(2-Pyridazin-3-Ylethylidene)Hydrazino]Propanenitrile and Glyoxal in the Presence of Sc(OTf)3

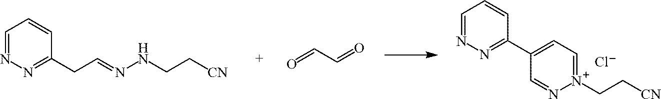

A 10 mL vial was charged with glyoxal (1.26 g, 8.72 mmol, 2.0 eq., 40% w/w in H₂O), hydrochloric acid (139 mg, 1.22 mmol, 1.2 eq. 32% w/w in H₂O) and Scandium(III) trifluoromethanesulfonate (254 mg, 0.52 mmol, 0.5 eq.). 3-[2-(2-Pyridazin-3-ylethylidene)hydrazino]propanenitrile (247 mg, 1.04 mmol, 80%) was added in a single portion and the resulting mixture was stirred at 45° C. for 2 h. The reaction mixture was sampled and analyzed by quantitative 1H NMR (in D₂O with Diethylene glycol diethyl ether as standard), indicating the title compound had been formed in 70% chemical yield. NMR data: ¹H NMR (400 MHz, D₂O) δ ppm: 10.26 (d, J=2.6 Hz, 1H), 9.98 (d, J=6.3 Hz, 1H), 9.41 (dd, J=5.1 Hz, 1.5 Hz, 1H), 9.25 (dd, J=6.3 Hz, 2.6 Hz, 1H), 8.60 (dd, J=8.7 Hz, 1.5 Hz, 1H), 8.07 (dd, J=8.7 Hz, 5.1 Hz, 1H), 5.28 (t, J=6.2 Hz, 2H), 3.47 (t, J=6.2 Hz, 2H).

### Example 33: Preparation of 3-(4-Pyridazin-3-Ylpyridazin-1-Ium-1-yl)Propanoic Acid Chloride Salt From 3-(4-Pyridazin-3-Ylpyridazin-1-Ium-1-yl)Propanenitrile Chloride Salt

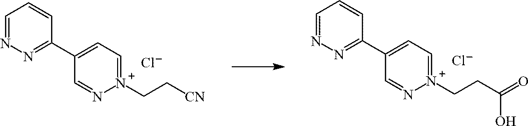 [SMILES: N#CCC[n+]1ccc(-c2cccnn2)cn1 ; [Cl-] ; O=C(O)CC[n+]1ccc(-c2cccnn2)cn1 ; [Cl-]]

3-pyridazin-3-ylpyridazin-1-ium-1-yl)propanenitrile chloride salt (17.9 g, 40.4 mmol, 55.8%) was stirred with hydrochloric acid (46.0 g, 0.404 mol, 10 eq, 32% w/w in H₂O) at 80° C. for 2.5 h. Water (31 g) was added and volatiles (HCl/Water azeotrope) were removed by rotary evaporation at 55° C. To remove excessive HCI as well as water, propionic acid (15.5 g) was added to the residue and the resulting mixture was evaporated to dryness to result in crude product as a black amorphous (glass-like) solid in 96% yield (24.9 g, purity=41.4%, quantitative 1H NMR in D₂O with 1-Methyl-2-pyridone as standard).

NMR data: ¹H NMR (400 MHz, D₂O) δ ppm: 10.13 (d, J=2.4 Hz, 1H), 9.95 (d, J=6.3 Hz, 1H), 9.34 (dd, J=5.1 Hz, 1.5 Hz, 1H), 9.15 (dd, J=6.3 Hz, 2.6 Hz, 1H), 8.57 (dd, J=8.7 Hz, 1.5 Hz, 1H), 8.04 (dd, J=8.7 Hz, 5.1 Hz, 1H), 5.18 (t, J=6.1 Hz, 2H), 3.29 (t, J=6.1 Hz, 2H).

### Example 34: Preparation of 3-(4-Pyrimidin-2-Ylpyridazin-1-Ium-1-yl)Propanenitrile Chloride Salt From 3-[2-(2-Pyrimidin-2-Ylethylidene)Hydrazino]Propanenitrile and Glyoxal in the Presence of ZrOCl₂*8H₂O

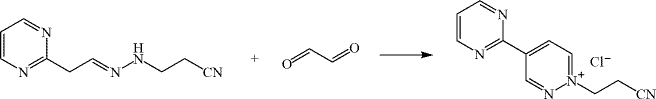

A 10 mL vial was charged with glyoxal (0.579 g, 3.99 mmol, 2.0 eq., 40% w/w in H₂O), hydrochloric acid (0.274 g, 2.40 mmol, 1.2 eq. 32% w/w in H₂O), Zirconium(IV) oxychloride octahydrate (66 mg, 0.20 mmol, 10 mol%) and Methanol (1.6 mL). 3-[2-(2-pyrimidin-2-ylethylidene)hydrazino]propanenitrile (0.50 g, 1.98 mmol, 69.5%) was added in a single portion and the reaction mixture was stirred for 4 h at 0-5° C. then for 2 h at room temperature. The reaction mixture was sampled and analyzed by quantitative 1H NMR (in D₂O with Diethylene glycol diethyl ether as standard), indicating the title compound had been formed in 85% chemical yield.

3-Methyl-1-butanol (2 mL) was added and the mixture was stirred at 45° C. for 30 min. During this time precipitation of the title compound was observed. After cooling to room temperature, the mixture was filtered. The brown solid (0.50 g) was analyzed by quantitative 1H NMR (in D₂O with Diethylene glycol diethyl ether as standard), indicating the following composition: 46% 3-(4-pyrimidin-2-ylpyridazin-1-ium-1-yl)propanenitrile chloride salt, 25% 3-Methyl-1-butanol and water.

NMR data: ¹H NMR (400 MHz, D₂O) δ ppm: 10.36 (d, J=2.1 Hz, 1H), 10.01 (d, J=6.2 Hz, 1H), 9.37 (dd, J=6.2 Hz, 2.1 Hz, 1H), 9.12 (d, J=5.0 Hz, 2H), 7.77 (t, J=5.0 Hz, 1H), 5.31 (t, J=6.3 Hz, 2H), 3.50 (t, J=6.3 Hz, 2H).

### Example 35: Preparation of 3-(4-Pyrimidin-2-Ylpyridazin-1-Ium-1-yl)Propanoic Acid Chloride Salt From 3-(4-Pyrimidin-2-Ylpyridazin-1-Ium-1-yl)Propanenitrile Chloride Salt

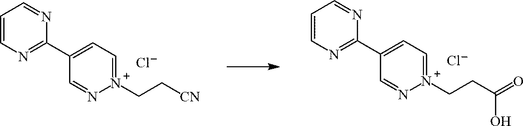 [SMILES: N#CCC[n+]1ccc(-c2ncccn2)cn1 ; [Cl-] ; O=C(O)CC[n+]1ccc(-c2ncccn2)cn1 ; [Cl-]]

3-Pyrimidin-2-ylpyridazin-1-ium-1-yl)propanenitrile chloride salt (1.66 g, 4.19 mmol, 62.5%) was stirred with hydrochloric acid (4.67 g, 25.6 mmol, 6 eq, 20% w/w in H₂O) at 110° C. for 9 h. After cooling to room temperature, the reaction mixture was concentrated to dryness by rotary evaporation to result in crude product as a brown-black solid in 70% yield (1.78 g, purity=43.7%, quantitative 1H NMR in D₂O with Diethylene glycol diethyl ether as standard).

NMR data: ¹H NMR (400 MHz, D₂O) δ ppm: 10.14 (d, J=2.1 Hz, 1H), 9.84 (d, J=6.2 Hz, 1H), 9.17 (dd, J=6.2 Hz, 2.1 Hz, 1H), 8.98 (d, J=5.0 Hz, 2H), 7.63 (t, J=5.0 Hz, 1H), 5.10 (t, J=6.1 Hz, 2H), 3.23 (t, J=6.1 Hz, 2H).

### Example 36: Preparation of 3-(4-Pyrimidin-4-Ylpyridazin-1-Ium-1-yl)Propanenitrile Chloride Salt From 3-[2-(2-Pyrimidin-4-Ylethylidene)Hydrazino]Propanenitrile

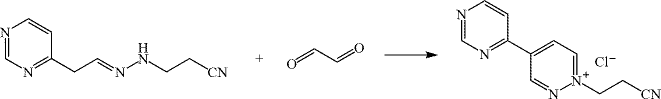

### Zirconium(IV) Oxychloride Octahydrate (0.317 G, 0.966 Mmol, 10 Mol%) Was Added to a Flask, Followed by Glyoxal (2.8 g, 19.3 Mmol, 2.0 Eq., 40% w/w in H₂O) and Hydrochloric Acid (1.36 g, 13 Mmol, 1.35 Eq. 35% w/w in H₂O) were Mixed (Solution 1)

### 3-(2-Pyrimidin-4-Ylethylidene)Hydrazino]Propanenitrile (3.0 G, 9.66 Mmol, 60.9%) and Methanol (4.7 G, 15.5 Eq.) were Mixed (Solution 2)

Solution 1 was cooled to 0° C. Methanol (1.56 g, 5 eq.) was dosed to solution 1 over 30 min and then mixture was stirred at 0-5° C. for 30 min.

Solution 2 was dosed to solution 1 over a period of 2 h while keeping the temperature at 0-5° C. After the end of addition, the reaction mixture was stirred for 1 h at 0-5° C. Acetonitrile (45 mL) was added and a suspension was observed. The mixture was filtered and the solid washed twice with acetonitrile (2 × 50 mL). The filtrate was concentrated under reduced pressure to obtain a brown solid. The solid was dissolved by adding water (20 mL) and extracting with ethyl acetate (4 × 100 mL). The aqueous phase was concentrated to dryness by rotary evaporation to result in the title compound as a black-brown solid in 69% yield (2.61 g, purity=63.1%, quantitative ¹H NMR in DMSO-d6 with two drops of D₂O with maleic acid as standard).

NMR data: ¹H NMR (400 MHz, DMSO-d6) δ ppm: 10.29 (d, J=2.06 Hz, 1 H), 10.14 (d, J=6.19 Hz, 1 H), 9.51 (s, 1 H), 9.37 (dd, J=6.19, 2.38 Hz, 1 H), 9.19 (br d, J=5.08 Hz, 1 H), 8.54 (d, J=4.44 Hz, 1 H), 5.21 (t, J=6.34 Hz, 2 H), 3.41 (t, J=6.34 Hz, 2 H).

### Example 37: Preparation of 3-[2-(2-Pyrimidin-4-Ylethylidene)Hydrazino]Propanenitrile From 4-[2-Pyrrolidin-1-Ylvinyllpyrimidine

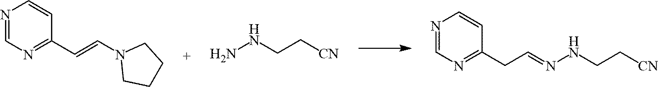

4-pyrrolidin-1-ylvinyl]pyrimidine (5.0 g, 27.1 mmol, 1.00 eq., 95% purity) was added to a solution of 3-hydrazinopropanenitrile (3.65 g, 42.8 mmol, 1.58 eq.) in ethanol (50 mL) cooled at 0-5° C. Next, trifluoroacetic acid (3.12 g, 27.1 mmol, 1.0 eq., 2.11 mL) was added dropwise to the above reaction mixture while maintaining the temperature below 10° C. After two hours, the mixture was concentrated in vacuo and purified over neutral alumina (0-4% MeOH in methyl tert-butyl ether) to obtain a yellow gum as an E/Z mixture (unassigned) in 47% yield (4.0 g, purity = 60.9%, quantitative 1H NMR in DMSO-d6 with 1,3,5-trimethoxybenzene as standard).

NMR data (mixture of E/Z-isomers): ¹H NMR (400 MHz, DMSO-d6) δ ppm 9.16 - 9.06 (m, 0.75 H), 8.74 - 8.68 (m, 1 H), 7.51 - 7.40 (m, 1 H), 7.20 (t, J=5.55 Hz, 0.75 H), 6.89 (t, J=4.84 Hz, 1 H), 6.80 (br s, 0.25 H), 6.60 (td, J=5.04, 1.35 Hz, 0.25 H), 3.67 - 3.59 (m, 2 H), 3.35 - 3.25 (m, 0.5 H), 3.25 - 3.15 (m, 1.5 H), 2.74 - 2.61 (m, 2 H).

### Example 38: Preparation of 4-[2-Pyrrolidin-1-Ylvinyl]Pyrimidine From 4-Methylpyrimidine

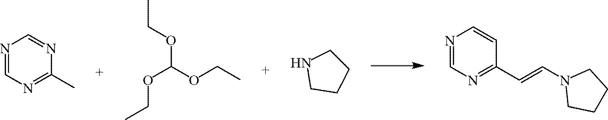

A 100 mL autoclave was charged with 4-methylpyrimidine (5 g, 52 mmol), pyrrolidine (1.9 g, 26 mmol, 0.5 eq.), triethyl orthoformate (6.3 g, 42 mmol, 0.8 eq.) and 2,6-Di-tert-butyl-4-methylphenol (230 mg, 1 mmol, 2 mol%). The mixture was heated at 155° C. for 4 h. After cooling to room temperature, the reaction mixture was concentrated in vacuo and purified over silica gel (20-35% ethyl acetate in cyclohexane) to obtain a light yellow solid in 44% yield (3.0 g, purity = 68% based on quantitative ¹H NMR in DMSO-d6 with 1,3,5-trimethoxybenzene as standard).

NMR data: ¹H NMR (400 MHz, DMSO-d6) δ ppm 8.58 (s, 1 H), 8.16 (d, J=5.62 Hz, 1 H), 8.00 (d, J=12.96 Hz, 1 H), 6.94 - 6.82 (m, 1 H), 4.95 (d, J=12.96 Hz, 1 H), 3.47 - 3.16 (m, 4 H), 1.87 (m, 4 H).

### Example 39: Preparation of 3-(4-Pyrimidin-4-Ylpyridazin-1-Ium-1-yl)Propanoic Acid Chloride Salt From 3-(4-Pyrimidin-4-Ylpyridazin-1-Ium-1-yl)Propanenitrile Chloride Salt

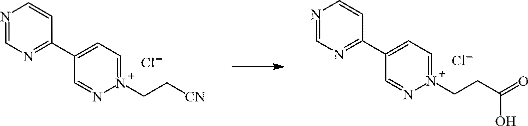 [SMILES: N#CCC[n+]1ccc(-c2ccncn2)cn1 ; [Cl-] ; O=C(O)CC[n+]1ccc(-c2ccncn2)cn1 ; [Cl-]]

3-pyrimidin-4-ylpyridazin-1-ium-1-yl)propanenitrile chloride salt (1.58 g, 4.03 mmol, 63.1%) was stirred with hydrochloric acid (6.29 g, 60.4 mmol, 15 eq, 35% w/w in H₂O) at 80° C. for 1 h. The reaction was cooled to room temperature to result in crude product in 88% yield (6.79 g, purity=14.1%, quantitative 1H NMR in D₂O with maleic acid as standard).

NMR data: ¹H NMR (400 MHz, D₂O) δ ppm: 9.92 (d, J=2.06 Hz, 1 H), 9.75 (d, J=6.19 Hz, 1 H), 9.28 (s, 1 H), 8.99 (dd, J=6.27, 2.46 Hz, 1 H), 8.96 (d, J=5.55 Hz, 1 H), 8.30 (dd, J=5.71, 1.27 Hz, 1 H), 4.95 (t, J=6.03 Hz, 2 H), 3.03 - 3.11 (m, 2 H).

1. A process for the preparation of a compound of formula (I) or an agronomically acceptable salt or zwitterionic species thereof: 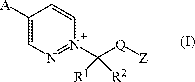
wherein
A is a 6-membered heteroaryl selected from the group consisting of formula A-I to A-VII below 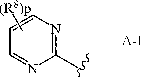 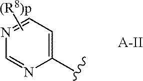 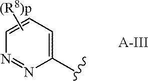 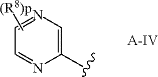 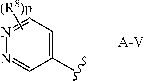 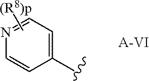 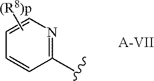
wherein the jagged line defines the point of attachment to the remaining part of a compound of formula (I), p is 0, 1 or 2; and
R¹ is hydrogen;
R² is hydrogen;
Q is (CR^{1a}R^{2b})ₘ;
m is 1;
each R^{1a} and R^{2b} are hydrogen:
Z is selected from the group consisting of —CN, —CH₂OR³, —CH(OR⁴)(OR^{4a}), —C(OR⁴)(OR^{4a})(OR^{4b}), —C(O)OR¹⁰, —C(O)NR⁶R⁷ and —S(O)₂OR¹⁰; or
Z is selected from the group consisting of a group of formula Zₐ, Z_{b}, Z_{c}, Z_{d}, Zₑ and Z_{f} below 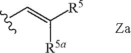 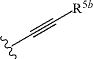 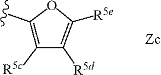 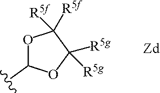 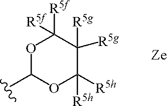 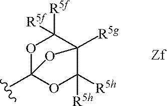
wherein the jagged line defines the point of attachment to the remaining part of a compound of formula (I); and
R³ is hydrogen or —C(O)OR¹⁰;
each R⁴, R^{4a} and R^{4b} are independently selected from C₁-C₆alkyl;
each R⁵, R^{5a}, R^{5b}, R^{5c}, R^{5d} R^{5e}, R^{5f}, R^{5g} and R^{5h} are independently selected from the group consisting of hydrogen and C₁-C₆alkyl;
each R⁶ and R⁷ are independently selected from the group consisting of hydrogen and C₁-C₆alkyl;
each R⁸ is independently selected from the group consisting of halo, —NH₂, methyl and methoxy;
R¹⁰ is selected from the group consisting of hydrogen, C₁-C₆alkyl, phenyl and benzyl; and
R^{10a} is selected from the group consisting of hydrogen, C₁-C₆alkyl, phenyl and benzyl; said process comprising:
reacting a compound of formula (IV); 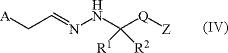
wherein A, Q, Z, R¹ and R² are as defined above;
with a compound of formula (V) or
a salt or an N-oxide thereof; 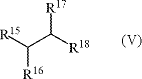
wherein
each R¹⁵, R¹⁶, R¹⁷ and R¹⁸ are independently selected from the group consisting of halogen, -OR^{15a}, -NR^{16a}R^{17a} and —S(O)₂OR¹⁰; and/or
R¹⁵ and R¹⁶ together are =O or =NR^{16a} and/or R¹⁷ and R¹⁸ together are =O or =NR^{16a}; or
R¹⁵ and R¹⁶ together with the carbon atom to which they are attached form a 3- to 6-membered heterocyclyl, which comprises 1 or 2 heteroatoms individually selected from nitrogen and oxygen; or
R¹⁵ and R¹⁷ together with the carbon atom to which they are attached form a 3- to 6-membered heterocyclyl, which comprises 1 or 2 heteroatoms individually selected from nitrogen and oxygen; and
each R^{15a} is independently selected from the group consisting of hydrogen and C₁-C₆alkyl;
each R^{16a} is independently selected from the group consisting of hydrogen and C₁-C₆alkyl;
each R^{17a} is independently selected from the group consisting of hydrogen and C₁-C₆alkyl;
to give a compound of formula (I).

2. (canceled)

3. (canceled)

4. (canceled)

5. A process according to claim 1, wherein p is 0.

6. A process according to claim 1, wherein A is selected from the group consisting of formula A-Ia to A-IIIa below, 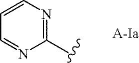 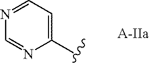 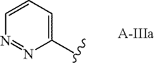
wherein the jagged line defines the point of attachment to the remaining part of a compound of formula (I).

7. A process according to claim 1, wherein Z is selected from the group consisting of —CN, —CH₂OH, —C(O)OR¹⁰, —S(O)₂OR¹⁰ and —CH═CH₂.

8. A process according to claim 1, wherein Z is —CN or —C(O)OR¹⁰.

9. A process according to claim 1, wherein the compound of formula (V) is a compound selected from the group consisting of a compound of formula (Va), (Vb), (Vc), (Vd), (Ve), (Vf), (Vg), (Vh), (Vj), (Vk) and (Vm), 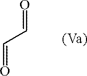 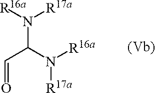 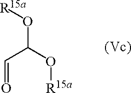 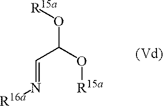 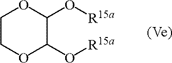 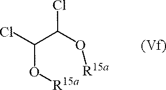 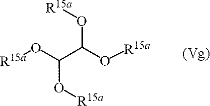 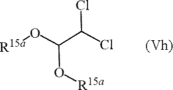 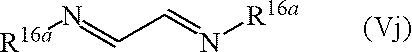 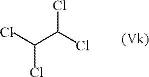 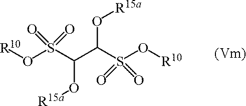
wherein each R¹⁰, R^{15a}, R^{16a} and R^{17a} are as defined in claim 1.

10. A process according to claim 1, wherein the compound of formula (V) is a compound of formula (Va), 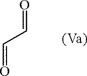
.

11. (canceled)

12. (canceled)

13. A process according to claim 1 wherein the compound of formula (IV) is produced by reacting a compound of formula (II): 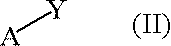
wherein A is as defined in claim 1;
Y is selected from the group consisting of a group of formula Y-I, Y-II and Y-III below 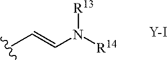 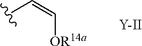 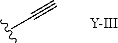
R¹³ and R¹⁴ are independently selected from the group consisting of hydrogen, C₁-C₆alkyl, C₁-C₆haloalkyl and phenyl; or
R¹³ and R¹⁴ together with the nitrogen atom to which they are attached form a 4- to 6-membered heterocyclyl ring which optionally comprises one additional heteroatom individually selected from nitrogen, oxygen and sulfur; and
R^{14a} is selected from the group consisting of hydrogen, C₁-C₆alkyl and —C(O)R^{14b};
R^{14b} is selected from the group consisting of hydrogen, C₁-C₆alkyl and C₁-C₆haloalkyl;
with a compound of formula (III): 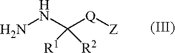
wherein R¹, R², Q and Z are as defined in claim 1, to give a compound of formula (IV); 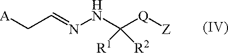
wherein A, Q, Z, R¹ and R² are as defined in claim 1.

14. A process according to claim 1 wherein the compound of formula (I) is further subjected to a hydrolysis, oxidation and/or a salt exchange to give an agronomically acceptable salt of formula (Ia) or a zwitterion of formula (Ib), 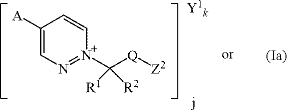 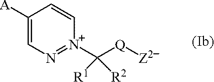
wherein Y¹ represents an agronomically acceptable anion and j and k represent integers that may be selected from 1, 2 or 3, and A, R¹, R² and Q are as defined in claim 1 and Z² is —C(O)OH or —S(O)₂OH.

15. (canceled)

16. A compound selected from the group consisting of a compound of formula (Ic) and a compound of formula (Id) or an agronomically acceptable salt thereof, 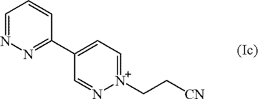 [SMILES: N#CCC[n+]1ccc(-c2cccnn2)cn1] 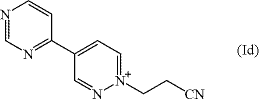 [SMILES: N#CCC[n+]1ccc(-c2ccncn2)cn1]
.

17. A compound of formula (IV) 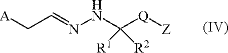
wherein A, Q, Z, R¹ and R² are as defined in claim 1.

18. Use of a compound of formula (II) for preparing a compound of formula (I) 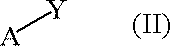
wherein A and Y are as defined in claim 1.

19. A compound of formula (II-a) 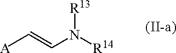
wherein A is a 6-membered heteroaryl selected from the group consisting of formula A-I, A-II, A-III, A-IV, A-V and A-VII below 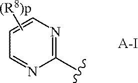 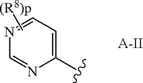 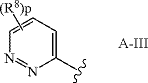 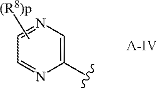 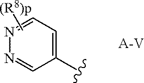 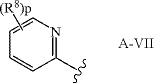
wherein the jagged line defines the point of attachment to the remaining part of a compound of formula (I), p and R⁸ are as defined in claim 1; R¹³ and R¹⁴ are independently selected from the group consisting of C₂-C₆alkyl, C₁-C₆haloalkyl and phenyl; or
R¹³ and R¹⁴ together with the nitrogen atom to which they are attached form a 4- to 6-membered heterocyclyl ring which optionally comprises one additional heteroatom individually selected from nitrogen, oxygen and sulfur.

20. (canceled)

21. A process according to claim 13 wherein the compound of formula (II) wherein Y is Y-I, is produced by:
reacting a compound of formula (VI) 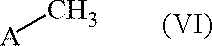
with a compound of formula (VII) 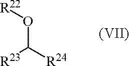
wherein R²² is C₁-C₆alkyl;
R²³ and R²⁴ are independently selected from the group consisting of C₁-C₆alkoxy and -NR²⁵R²⁶;
R²⁵ and R²⁶ are independently selected from C₁-C₆alkyl; or
R²⁵ and R²⁶ together with the nitrogen atom to which they are attached form a 4- to 6-membered heterocyclyl ring which optionally comprises one additional heteroatom individually selected from nitrogen, oxygen and sulfur;
and a compound of formula (VIII) 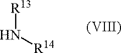
to produce a compound of formula (II) 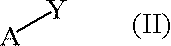
.

22. (canceled)

23. Use of a compound of formula (VI) for preparing a compound of formula (I) 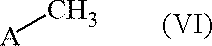
wherein A is as defined in claim 1.

24. Use of a compound of formula (III) for preparing a compound of formula (I) 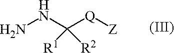
wherein R¹, R² Q and Z are as defined in claim 1.
